(19) United States
(12) Patent Application Publication
(10) Pub. No.: US 2025/0114298 A1
(43) Pub. Date: Apr. 10, 2025

(54) ZWITTERIONIC HYDROGEL DEPOT FOR EXTENDED ANTIGEN AND VACCINE DELIVERY

(71) Applicant: The Hong Kong University of Science and Technology, Hong Kong (CN)

(72) Inventors: Jin Teng CHUNG, Hong Kong (CN); Ying CHAU, Hong Kong (CN); Chi Ming Laurence LAU, Hong Kong (CN)

(21) Appl. No.: 18/907,743

(22) Filed: Oct. 7, 2024

Related U.S. Application Data
(60) Provisional application No. 63/588,002, filed on Oct. 5, 2023.

(51) Int. Cl. A61K 9/00; A61K 9/06; A61K 39/00; A61K 39/385; A61P 37/04; C08B 37/02

(52) U.S. Cl. CPC A61K 9/0024; A61K 9/06; A61K 39/385; A61P 37/04; C08B 37/0021; A61K 2039/55583

(57) ABSTRACT

The subject invention pertains to a long-term antigen depot that can bypass FBR and engage DCs to mature and migrate to lymph nodes to activate antigen-specific T-cell activations. Leveraging on the immunomodulatory properties of exogenous polysaccharides and the anti-fouling characteristics of zwitterionic phosphorylcholine (PC) polymer, the invention features a PC functionalized dextran (PCDX) hydrogel for long-term antigen delivery. PCDX in both injectable scaffold and microparticles (MPs) forms effectively evades FBR and provides slow release of antigens resulting in local enrichment of CD11c⁺ DCs at the MPs injection sites. DC cultured on PCDX exhibits strong immunogenic activation with high CD86, CD40, MHC-I/peptide complex. PCDX also generates DC with propensity in migration to lymph nodes, as well as antigen presentation to trigger both CD4+ and CD8+ arms of T-cell responses. Besides cellular responses, PCDX can also induce potent humoral responses, with high levels of antigen specific IgG1 and IgG2a by day 28. Overall, PCDX provides long-term delivery of antigens for vaccine development.

### CROSS-REFERENCE TO RELATED APPLICATION

This application claims the benefit of U.S. Provisional Application Ser. No. 63/588,002, filed Oct. 5, 2023, which is hereby incorporated by reference in its entirety including any tables, figures, or drawings.

### BACKGROUND OF THE INVENTION

A potent vaccine involves the sustained co-delivery of antigens and adjuvants to the professional antigen presenting cells (APCs). Dendritic cells (DCs) are the key APCs that bridge innate and adaptive immunity by migrating to nearby lymph nodes and presenting antigens on the MHC-molecules to activate both CD8+ and CD4+ T-cells [1-3]. Most antigens are proteins, which delivery, however, is greatly limited by diffusion, degradation, and systemic clearance before they are able to reach their target DC subtypes in the skin [4-6]. In early delivery strategies, antigens were adsorbed to insoluble aluminium salts (alum), oil-in-water emulsions or liposomes, to form depots and achieve sustained release at the injection sites [7,8]. Inspired by the natural infection mechanisms of pathogens, the next-generation vaccines consist of biomaterials-based polymeric scaffolds, which are designed to act as localized depots for the sustained delivery of antigens to DCs. Such depots can better mimic the kinetics of pathogenic and inflammatory cues by generating a concentration gradient of antigens and adjuvants that can more effectively potentiate DC responses to produce adaptive immunity against the “quasi pathogens” [9,10]. For instance, PLGA-based scaffold was developed and implanted subcutaneously to mimic infections and to deliver antigen and adjuvants, GM-CSF and CpG, to recruit DCs. Possible source of antigens may include well-defined tumor-associated antigens or tumor lysates, the latter being found to elicit effective anti-tumor responses [11]. In addition, exogenous polysaccharides, which exhibit immunomodulatory propensity, are also good candidates in preparing antigen depots to mimic infections [12,13]. For instance, the immunogenic chitosan and mannan combination has been formulated into bulk hydrogel for sustained antigen delivery. The self-adjuvanticity provides stronger humoral responses than alum in vivo [14].

While immunomodulatory depots are optimal for antigen delivery, the prolonged subcutaneous biomaterial implants often induce foreign body responses (FBR). Macrophages are one of the cell players in FBR and, in contrast to DCs, macrophages exhibit greater phagocytic capacity, adhesive characteristics, and are able to fuse with infiltrated monocytes to form multinucleated foreign body giant cells (FBGCs) to facilitate local clearance of large foreign bodies [15-17]. With the interceptions of phagocytic clearance of antigen cargoes by macrophage and depot isolation by fibroblasts during FBR, the prolonged release of antigens from depots are largely hampered and the roles of DCs in processing and presenting antigens to lymphocytes are greatly downplayed in triggering robust adaptive immune responses. Therefore, there is a need for an extended vaccine delivery method that can bypass FBR and stimulate a durable and potent humoral and cell-mediated immunity.

### BRIEF SUMMARY OF THE INVENTION

The subject invention discloses a zwitterionic compound of formula I, Formula (I)

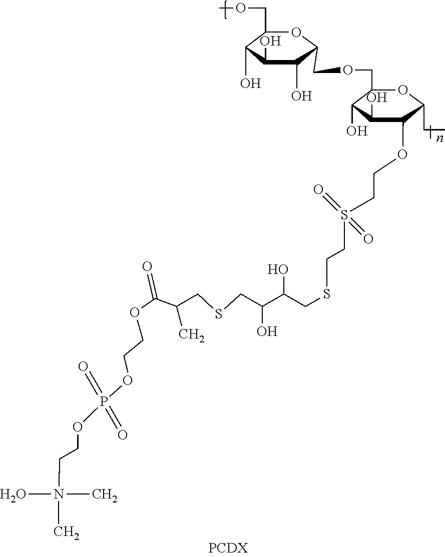

where the zwitterionic compound is PCDX.

In one aspect, PCDX can be synthesized from the conjugation of phosphorylcholine moieties with chemically modified DX, in the forms of aldehyde, vinyl sulfone, acrylate, methacrylate, at various degree of modifications (DM) of about 1 to about 20%.

PCDX product can be functionalized with pendant crosslinking moieties, such as acrylate, methacrylate, vinyl sulfone, aldehyde, amine, and thiol groups at various degree of modifications of about 1 to about 20% to obtain an activated hydrogel precursor of PCDX, which is crosslinked to form an injectable PCDX hydrogel. The injectable PCDX hydrogel can be used as a hydrogel depot, which includes, but is not limited to, bulk hydrogel and hydrogel microparticles (MPs). The hydrogel depot can be used as a carrier that comprises vaccine components, which include, but are not limited to, one or more antigens. The hydrogel depot is injected under the skin of a subject for the sustained release of the antigens to induce effective immune responses in the subject. The hydrogel depot is used as an antigen depot and as an adjuvant.

In preferred embodiments, both the injectable scaffold and microparticles (MPs) forms of the hydrogel depot effectively evade FBR when injected under the skin of a subject. Furthermore, when injected subcutaneously in a subject, the PCDX hydrogel significantly induces the expression of markers selected from the group consisting of DCs markers CD86 and CD40, lymph node marker CCR7, and mannose receptor CD206.

In another aspect, provided herein is a method for extended vaccine delivery, where an effective amount of MPs, comprising a PCDX hydrogel that comprises one or more antigens, and optionally an excipient, including cytokines, natural and synthetic vaccine adjuvants, is injected subcutaneously in a subject for the sustained release of the one or more antigens to induce effective immune responses in the subject.

In certain embodiments, an effective amount of bulk hydrogel comprising a PCDX hydrogel that comprises one or more antigens, and optionally an excipient, is injected subcutaneously in a subject for the sustained release of the one or more antigens to induce effective immune responses in the subject.

In certain embodiments, the one or more antigens include, but are not limited to, a protein, a nucleic acid, a sugar, a lipid, or a glycoprotein, and combinations thereof.

In certain embodiments, the subject is a mammal, and the mammal is a human.

In certain embodiments, the MP has a diameter that ranges from about 2 to about 500 μm. In certain embodiments, the MP is used as an antigen depot and as a self adjuvant. In preferred embodiments, the MP effectively evade FBR when injected under the skin of a subject.

In further embodiments, the MP significantly induces the expression of markers selected from the group consisting of DCs markers CD86 and CD40, lymph node marker CCR7, and mannose receptor CD20, as well as other pro-inflammatory markers such as CD80/CD83, MHC-II, MHC-I, and secreted cytokines, e.g. IFN-g, TNF-a, IL-6, IL-1b.

### BRIEF DESCRIPTION OF THE DRAWINGS

FIGS. 1A-1D illustrate the physical characterizations of DX and derivative hydrogels. FIGS. 1A-1B show storage modulus of hydrogel disc prepared at 30% wt/v polymer concentrations under frequency and strain sweeps. FIG. 1C shows surface charge measurements of hydrogel microparticles at various pHs. FIG. 1D shows the mass swelling ratio of non-hydrolysable DX and derivatives hydrogel spheres incubated in PBS buffer at 37° C.

FIGS. 2A-2H illustrate foreign body response assays in vitro and in vivo. FIG. 2A shows the viability and adhesion of NIH 3T3 fibroblasts, RAW264.7 macrophages and JAWSII dendritic cell lines cultured on PCDX, DX, DEDX and CMDX hydrogels surface as compared to control tissue culture plate (TCP). Cells were cultured for 72 hrs and stained with Live/Dead viability dye. For adhesion assay, cultures were rinsed with warm PBS three times prior to fluorescence imaging. Scale bar represents 100 μm. FIG. 2B shows the quantification of fluorescence signals from cells cultured on hydrogels after PBS rinsing (N=6). The graph was represented as mean and s.d. FIG. 2C shows the relative protein adsorption percentage of F-fibrinogen on hydrogels and TCP surface. The graph is represented as mean and s.d. (N=6, * is in comparison to TCP surface, where p<0.05). FIG. 2D shows the skin condition of injection sites and area of in-situ crosslinked hydrogels are marked with the dotted lines. The hydrogel scaffolds were extracted on day 7 post-injection, showing different sizes and extent of fibrotic encapsulation and angiogenesis in DX, PCDX, CMDX and DEDX. FIG. 2E shows representative hematoxylin and eosin (H&E) staining images of dorsal skin tissues at the injection site on day 7 post-injection. Scale bar represents 300 μm. FIG. 2F shows the surface markers profile of RAW264.7 cultured on bulk hydrogel surface for 24 hr. Graph is presented as geometric mean fluorescence intensity ratio to non-treated cells. (N=4, * is in comparison to cells on TCP surface and treated with 1 μg/mL, where p<0.05). FIGS. 2G-2H show the production of cytokines IL-6 and TNF-α from RAW264.7 after 24 hr culture on hydrogels and TCP with 1 μg/mL LPS treatment. (N=4, * is in comparison to cells on DX surface, where p<0.05).

FIGS. 3A-3F illustrate hydrogel microparticles (MPs) characterizations. FIG. 3A shows the size and morphology of hydrogel MPs viewed and estimated under microscope. The mean diameter and s.d. are summarized in the adjacent table (FIG. 3A, right side). The MPs and JAWSII interactions at 24-hour co-culture were also observed under confocal fluorescence microscope. The nucleus (blue) was stained with Hoechst 33342, lysosomes (red) with lysotracker and MPs (green) chemically conjugated with FITC on DX and derivatives polymer backbones. Scale bar represents 50 μm. FIG. 3B shows the release profile of FITC-conjugated ovalbumin (F-OVA) encapsulated in degradable -OAc formulations of DEDX, DX, PCDX and CMDX hydrogels. FIG. 3C shows the viability of JAWSII at 24-hour co-culture with MPs examined with propidium iodide staining and flow cytometric analysis. The graph is represented as mean cell population % and s.d. (N=6). FIG. 3D shows the uptake level of OVA released from MPs by JAWSII at 24-hour co-culture. The graph is presented as geometric mean fluorescence intensity (MFI) as ratio relative to non-F-OVA treated cells and s.d. (N=6, + is in comparison to DX, p<0.05). FIGS. 3E and 3F show the MHC-II and MHC-I SIINFEKL expression levels on JAWSII at 24-hour co-culture with OVA releasing MPs. The graph is presented as geometric mean fluorescence intensity as ratio relative to non-OVA treated cells and s.d. (N=6, * is in comparison to TCP+OVA, p<0.05). PI: propidium iodide, MFI: mean fluorescence intensity, NT: no treatment.

FIG. 4 illustrates in vitro JAWSII maturation phenotypes. The effects of charge modifications on DX, namely anionic CMDX, cationic DEDX, and zwitterionic PCDX MPs on the expression levels of DC maturation markers CD40 and CD86, and lymph node homing markers CCR7, mannose receptors CD206. The production of cytokines IL-6, TNF-α and IL-1β in culture medium supernatants were also measured. JAWSII were cultured with MPs for 24-hour and for positive control, JAWSII were cultured on TCP and treated with 1 μg/mL LPS for 24-hour. For cell surface marker expression levels, the graphs are presented as geometric mean fluorescence intensity ratio to naïve cells cultured on TCP, mean and s.d. For cytokine levels, the graphs are presented as mean cytokine concentration and s.d. (N=6, * is in comparison to native DX MPs, p<0.05).

FIGS. 5A-5D illustrate in vivo FBR of MPs. FIG. 5A shows the dissections of skin at the MPs injection site of immunocompetent C57BL\6 mice on day 7, 14, and 21 to monitor the development of FBR in CMDX, DEDX, DX, and PCDX treatment groups. The MPs injected areas were circles with white dotted lines. In the negative control, bolus group, animals were also injected subcutaneously with same dose of OVA dissolved in PBS buffer (350 μg OVA per animal. FIGS. 5B-5D show the skin and MPs harvested on day 7, 14, and 21 and digested with collagenase and dextranase enzyme cocktails to prepare single cell suspensions for flow cytometry analysis. F4/80⁺ CD206⁺ and F4/80⁺ CD86, representing M2 and M1 macrophages populations, as well as activated DCs CD11c⁺ CD86⁺, and migratory DCs CD11c⁺ CCR7⁺ populations were gated. The graph is presented as average gated cell population percentage and s.d. (N=5, * is in comparison to DX for each cell population, p<0.05, and **p<0.001).

FIGS. 6A-6D illustrate biodistribution of OVAIR signals with time. FIGS. 6A and 6C show the average quantitative fluorescence signals of injected OVAIR encapsulated in MPs or bolus solution with time. The graphs are presented as mean and s.d. (N=5). Fluorescence signals were quantified with ImageJ software. FIGS. 6B and 6D show representative heat maps of the IR scanning of excised dorsal skin at the injection sites and inguinal lymph nodes at day 7, 14 and 21. Animals in sham group were injected with PBS and the tissue samples were used as blank to define signals threshold. Image was visualized with Image Studio Lite Version 5.2 software.

FIGS. 7A-7G illustrate antigen-specific cellular responses of controlled release of OVA from MPs. FIG. 7A shows the level of CD11c⁺ population percentage in ILN harvested on day 7 post-injection, from animals treated with CMDX, DEDX, DX, PCDX MPs encapsulating OVA in comparison to bolus OVA treatment. FIGS. 7B and 7C show the frequency of activated CD11c⁺ CD86⁺, and CD11c⁺ CD40⁺ subsets in ILN on day 7. FIGS. 7D and 7E show the population distribution of T-lymphocytes in ILN and spleen on day 7. CD3⁺, CD3⁺ CD4⁺, CD3⁺ CD8⁺ cells were gated, and the population percentages were compared. FIGS. 7F and 7G show the ratio of activated T-cells, CD3⁺ CD25⁺ subsets to total CD3⁺ T-cells in ILN and spleen. All graphs are presented as mean and s.d. (N=6, * is in comparison with bolus OVA as negative control, p<0.05 and **p<0.001).

FIGS. 8A-8D-FIGS. 8A and 8B show the concentrations of pro-inflammatory cytokines, IL-6 and TNF-α in serum measured 7 days post-treatment. FIGS. 8C and 8D show the anti-OVA IgG1 and IgG2a antibodies titers measured with ELISA on day 7, 14, and 28 for all treatment groups. All graphs are presented as mean and s.d. (N=6, * is in comparison with bolus as negative control, p<0.05).

FIGS. 9 (I)-(III) illustrate the synthesis scheme of DXVS and zwitterionic PCDX. FIG. 9 (I) shows DX activated with vinyl sulfone group (VS) by reacting DX at 5% wt/V polymer concentration with DVS, at molar ratio DVS:OH of 1.2:1. Reaction occurred in 0.02M NaOH and stopped with 0.02M HCl after various time points to control degree of modifications (DM). FIG. 9 (II) shows MPC-SH synthesized by reacting MPC to DTT at molar ratio MPC:DTT of 1:6 in 0.05M phosphate buffer, pH 6.4 overnight at room temperature. The product was extracted via acetone precipitation. FIG. 9 (III) shows PC synthesized by reacting MPC-SH with DXVS at molar ratio VS:SH of 1:3 in 0.1M phosphate buffer of pH 7.4 for 2 hour at room temperature.

FIG. 10 illustrates the relationship between the DM of VS on DX with time at 5% wt/V DX polymer concentration and 0.02M NaOH. Reactions were stopped with 0.02M HCl.

FIGS. 11A-11D illustrate the chemical structure and ¹H-NMR spectra. FIG. 11A shows VS functionalized DX, FIG. 11B shows MPC, FIG. 11C shows thiolated MPC, MPC-SH and FIG. 11D shows DX modified with PC.

FIGS. 12 (IV-VII) illustrate the synthesis scheme -OAc functionalized DX and charge derivatives. FIG. 12 (IV-VII) show DX and charge derivatives, CMDX, DEDX, and PCDX activated with acryloyl group (-OAc) by reacting polysaccharides at 5% wt/V polymer concentration with acryloyl chloride, at a molar ratio OAc:OH of 0.5:1 in ddH₂O. 5M NaOH was added dropwise to maintain reaction pH at 9 to 10 for 1 hour, in ice bath. Reaction was stopped with 6M HCl addition to pH 5. All-OAc functionalized products were purified with dialysis and lyophilized.

FIGS. 13A-13D illustrate the chemical structure and ¹H-NMR spectra of -OAc functionalized dextran (DX-OAc) (FIG. 13A), carboxymethyl dextran (CMDX-OAc) (FIG. 13B), diethyl aminoethyl dextran (DEDX-OAc) (FIG. 13C), and phosphorylcholine-decorated dextran (PCDX-OAc) (FIG. 13D).

FIGS. 14A-14B illustrate representative flow cytometry flow from C57BL\6 mice tissue samples post hydrogel injection, showing gating strategy for FIG. 14A) CD3+, CD4+, CD8+ T cell subsets and FIG. 14B) CD11c⁺ with activation markers CD86 and CCR7, as well as F4/80+ cells with CD206 and CD86, % total of each subsets are labelled in the graphs.

FIGS. 15A-15D illustrate scanning electron microscope images of DX (FIG. 15A), DEDX (FIG. 15B), CMDX (FIG. 15C), and PCDX (FIG. 15D). Bulk hydrogels were prepared with 40% wt/V DX-OAc, DEDX-OAc, CMDX-OAc and PCDX-OAc and crosslinked with DTT. Hydrogels were incubated overnight at 37° C., in humidified chamber to ensure complete gelation. Subsequently, hydrogels were allowed to swell in ddH₂O for 2 hr. The hydrogels were blotted dry with Kimwipe and compaction with centrifuge, 1000 rpm for 30 sec. Subsequently, hydrogels were subjected to snap freezing in liquid N₂ and lyophilization. The freeze-dried hydrogels were cut into smaller pieces of ˜ 2 mm and subjected to gold sputter coating (K575xd, Emitech Ltd). The hydrogels were imaged with scanning electron microscope (JSM-7100F JEOL) at 20 kV.

FIG. 16 illustrates the swelling behavior of degradable-OAc modified DX, CMDX, DEDX and PCDX bulk hydrogels.

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” means within an acceptable error range for the particular value as determined by one of ordinary skill in the art, which depends in part on how the value is measured, i.e., the limitations of the measurement system. In the context of compositions containing amounts of ingredients where the terms “about” are used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, the term “subject” refers to an animal, needing or desiring delivery of the benefits provided by a therapeutic compound. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

The subject invention pertains to a novel injectable, immunomodulating polysaccharide-based depot system for the sustained release of antigens to induce effective immune responses and prolong antigen delivery to DCs, while effectively evading FBR.

In a first aspect of the subject invention, disclosed herein is a novel injectable polysaccharide-based depot system. The depot system comprises a zwitterionic PCDX hydrogel for extended antigen delivery. In certain embodiments, the PCDX hydrogel depot comprises two dosage forms, including, but not limited to, bulk hydrogel and MPs. PCDX can effectively engage DCs in vitro and in vivo while effectively evade FBR. In preferred embodiments, the charge-charge interactions of the zwitterionic moieties of PCDX resist rapid hydrogel swelling and the additional physical crosslinks slow down the antigen release.

In certain embodiments, PCDX can be synthesized from the conjugation of phosphorylcholine moieties with chemically modified DX, in forms including, but not limited to, aldehyde, vinyl sulfone, acrylate, methacrylate, at various degree of modifications (DM) of about 1 to about 20%. In preferred embodiments, PCDX can be synthesized from the conjugation of phosphorylcholine moieties with chemically modified DX, in forms including, but not limited to, aldehyde, vinyl sulfone, acrylate, methacrylate, at various degree of modifications (DM) of about 4 to about 15%. In more preferred embodiments, PCDX can be synthesized from the conjugation of phosphorylcholine moieties with chemically modified DX, in forms including, but not limited to, aldehyde, vinyl sulfone, acrylate, methacrylate, at various degree of modifications (DM) of about 8%.

In certain embodiments, PCDX hydrogel MPs can be utilized as a carrier for long-term delivery of protein-based antigens in various vaccine development. In some embodiments, sustained antigen release from the PCDX hydrogel effectively induces higher antigen presentation on MHC-I and MHC-II, triggering both CD8+ and CD4+ T-cell responses in vivo. In certain embodiments, the PCDX hydrogel induces DCs to express moderate level of maturation markers CD86 and CD40, and lymph node homing marker CCR7 at a significantly higher expression level. In certain embodiments, the PCDX hydrogel promotes potent and durable humoral responses with high level of anti-OVA antibodies at extended days post-immunization.

Subcutaneous depots are often susceptible to foreign body responses (FBR) dominated by macrophage clearance and fibrotic encapsulation, resulting in limited antigen delivery to target dendritic cells (DCs) that bridge innate and adaptive immunity. In preferred embodiments, the zwitterionic polysaccharide-based immunogenic depots of the subject invention effectively circumvent FBR while engaging specific innate immune cell populations locally at the injection site and eliciting robust immune response.

In certain embodiments, the hydrogel carriers are used in conjunction with vaccine components in pharmaceutical settings to enable extended vaccine delivery within the patients.

In preferred embodiments, the controlled release of antigen from the injectable depots confers long-lasting immune responses in a subject.

In some embodiments, the PCDX hydrogel depot provides long-term vaccine delivery while replacing multiple booster shots.

In a second aspect of the subject invention, disclosed herein is a method for extended vaccine delivery, comprising (a) providing PCDX hydrogel MPs, (b) injecting subcutaneously in a subject an effective amount of the MPs, which comprise one or more antigens, and optionally an excipient, where the one or more antigens provide the sustained release of the one or more antigens to induce effective immune responses in the subject.

In embodiments, the subject is a mammal and preferably the mammal is a human.

In certain embodiments, the MP has a diameter that ranges from about 2 to about 500 μm. In certain embodiments, the MP has a diameter that ranges from about 5 to about 100 μm.

In certain embodiments, the MP has a diameter that ranges from about 68 to about 80 μm. In certain embodiments, the MP has a diameter that of about 70 μm. In preferred embodiments, the MP has a diameter of about 72.4±3.1 μm.

In certain embodiments, the PCDX hydrogel depot can be used as a self-adjuvant that interacts and activates local APCs.

In some embodiments, the one or more antigens include, but are not limited to, a protein, a nucleic acid, a sugar, a lipid, or a glycoprotein, and combinations thereof. In further embodiments, the antigen includes, but is not limited to, a peptide, a polypeptide, a phospholipid, a cellular organelle, a bacterial component, a viral component, an inactivated virus, a recombinant protein, a recombinant nucleic acid, a synthetic nucleic acid, a synthetic peptide or polypeptide or protein, an allergen, a lectin, an organic compound, a plant component, an animal component, a synthetic polymer, a graphene-containing molecule, an antibody, and combinations thereof.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

Regardless of the route of administration selected, the composition may be formulated into pharmaceutically acceptable dosage form by conventional methods known to those of skill in the art. The composition may be formulated for administration in any convenient way for use in human or veterinary medicine, by analogy with other pharmaceuticals.

The compositions can further comprise one or more pharmaceutically acceptable carriers, and/or excipients, and can be formulated into preparations, for example, semi-solid or liquid forms, such as solutions or injections.

The formulations may conveniently be presented in unit dosage form and may be prepared any methods well known in the art of pharmacy. The amount of compound which can be combined with a carrier material to produce a single dosage form will vary depending upon the subject being treated, the particular mode of administration. The data obtained from cell culture assays and animal studies may be used in formulating a range of dosage for use in human. The amount of an active ingredient which can be combined with carrier material to produce a single dosage form will usually be the amount of the compound which produces a therapeutic effect. Usually, out of one hundred percent, this amount will range from about 1 wt % to about 99 wt % of active ingredient, preferably from about 5 wt % to about 70 wt %, most preferably from about 10 wt % to about 30 wt %.

As used herein, a “pharmaceutical” refers to a compound manufactured for use as a medicinal and/or therapeutic drug.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the adjuvant composition, carrier or excipient use in the subject compositions may be contemplated.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic assays or imaging techniques for detecting tumor sizes known in the art. In some embodiments of the invention, the method comprises administration of the compounds at several time per day, including but not limiting to 2 times per day, 3 times per day, and 4 times per day.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Materials and Methods

Three types of polysaccharides, dextran (DX, Mw=40 kDa, D1662 Sigma-Aldrich, US), carboxymethyl dextran (CMDX, Mw=40 kDa, C3251 TCI Chemicals, Japan), and diethylamino ethyl dextran (DEDX, Mw=40 kDa, 9015-73-0 TdB Labs, Sweden) were obtained. Chemicals 2-methacryloyloxyethyl phosphorylcholine (MPC, 730114 Sigma-Aldrich, US), divinyl sulfone (DVS, V3700 Sigma-Aldrich, US), DL-dithiothreitol (DTT, D9779 Sigma-Aldrich, US), and acrylol chloride (A0147, TCI Chemicals, Japan), IRDye® 800CW NHS ester (LI-COR, US) were purchased. Biological reagents ovalbumin (OVA, A7641 Sigma-Aldrich, US), fluorescein isothiocyanate conjugated ovalbumin (F-OVA, A9771 Sigma-Aldrich, US), recombinant murine GM-CSF (51048-MNAH SinoBiological, China), and lipopolysaccharide (LPS, L6529 Sigma-Aldrich, US) were obtained. Fluorescent imaging dyes, Live/Dead™ viability/cytotoxicity kit (L3224 ThermoFisher, US), LysoTracker™ Red DND-99 (L7528 ThermoFisher, US), and Hoechst 3328 (H1398 ThermoFisher, US) were purchased. Fluorescently labelled antibodies for flow cytometry, FITC anti-mouse I-Aκ (Aβκ) (MHC class II, clone 10-3.6), FITC anti-mouse CD86 (clone GL-1), PE anti-mouse CD40 (clone 3/23), APC anti-mouse CD197 (CCR7, clone 4B12), APC anti-mouse H-2K^{b} bound to SIINFEKL (MHC class I-SIINFEKL complex, clone 25-D1.16), FITC anti-mouse CD206 (MMR, clone C068C2), Alexa Fluor® 488 anti-mouse CD3 (clone 17A2), APC/Cyanine7 anti-mouse CD8b.2 (clone 53-5.8), Alexa Fluor® 647 anti-mouse CD4 (clone GK1.5), PE-Cy7 anti-mouse CD25 (clone 3C7), Alexa Fluor® 647 anti-mouse CD11c (clone N418), APC anti-mouse F4/80 (clone QA17A29) as well as isotype-matched controls: FITC mouse IgG2a κ isotype control (clone MOPC-173), FITC Rat IgG2 (clone RTK2758), APC Mouse IgG1 κ isotype control (clone MOPC-21), APC Rat IgG2a κ isotype control (clone RTK2758), PE Rat IgG2a κ isotype control (clone RTK2758), Alexa Fluor® 647 Rat IgG2b κ isotype control (clone RTK4530), APC/Cyanine7 Rat IgG1 κ isotype control (clone RTK2071), PE-Cy7 Rat IgG2b κ, and Alexa Fluor® 488 Rat IgG2b κ isotype control (clone RTK4530). All mAbs were purchased from Biolegend (US). ELISA kits for TNF-α (#430904), IL-6 (#431304), IL-1β (#432604), as well as biotinylated antibodies anti-OVA IgG1 (#406604) and IgG2a (#407104), were obtained from Biolegend. Dextranse from Chaetomium erraticum (D0443, Sigma-Aldrich, US) was purchased.

### Cells

NIH 3T3 (CRL1658™), JAWSII (CRL11904) and RAW264.7 (TIB-71™) cells were purchased from American Type Culture Collection (ATCC), US. JAWSII was cultured in α-MEM with ribonucleosides, deoxyribonucleosides, 4 mM L-glutamine, 1 mM sodium pyruvate and supplemented to 20% fetal bovine serum (FBS), 1% penicillin/streptomycin and 5 ng/ml GM-CSF. NIH 3T3 and RAW264.7 were maintained in DMEM supplemented with 10% FBS and 1% penicillin/streptomycin. All cell lines were maintained in a humidified incubator at 37° C. in 5% CO₂ and passaged every 3 to 5 days according to ATCC protocols.

Animals Female C57BL\6 mice (5 to 7 week) were provided by Laboratory Animal Facility (CWB) of The Hong Kong University of Science and Technology (HKUST) and maintained in pathogen-free conditions. Mice were housed in 12-h light/dark cycle and food and water ad libitum. All animal procedures were approved by the Animal Ethics Committee at HKUST and conducted in accordance with the guidelines of the Laboratory Animal Facility of HKUST.

### Synthesis and Characterizations of Dextran Derivatives as Microparticle (MP) Precursors

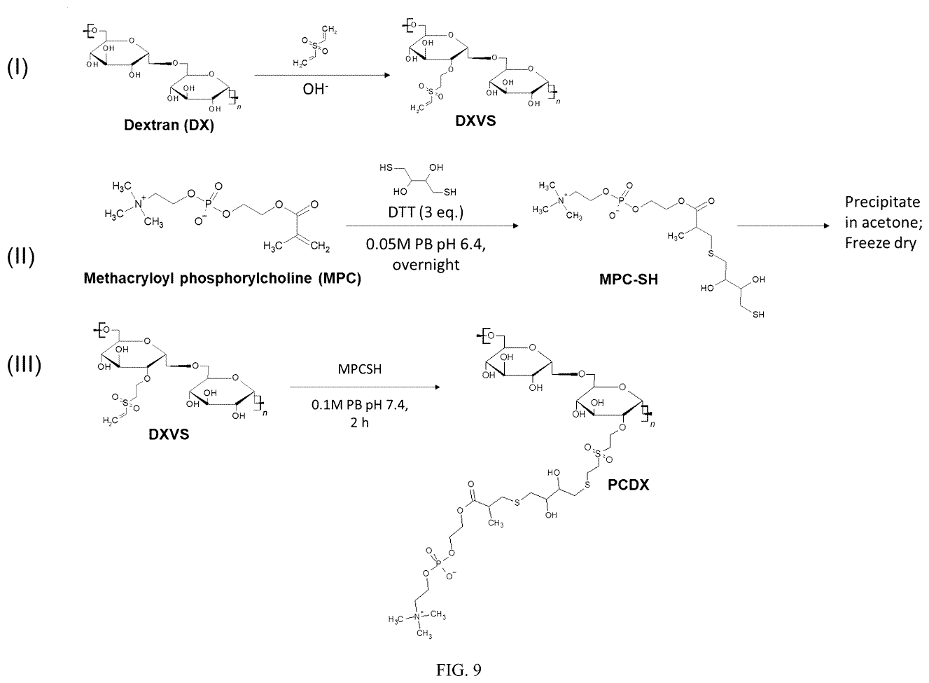

To synthesized zwitterionic PCDX, DX was first activated with VS and subsequently conjugated with thiolated PC, as shown in FIG. 9.

### VS Precursor Preparation.

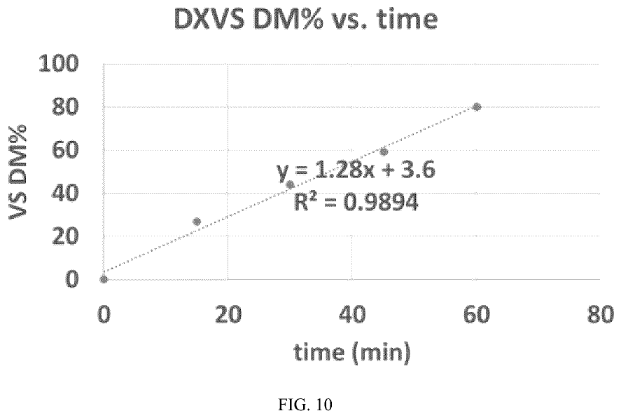

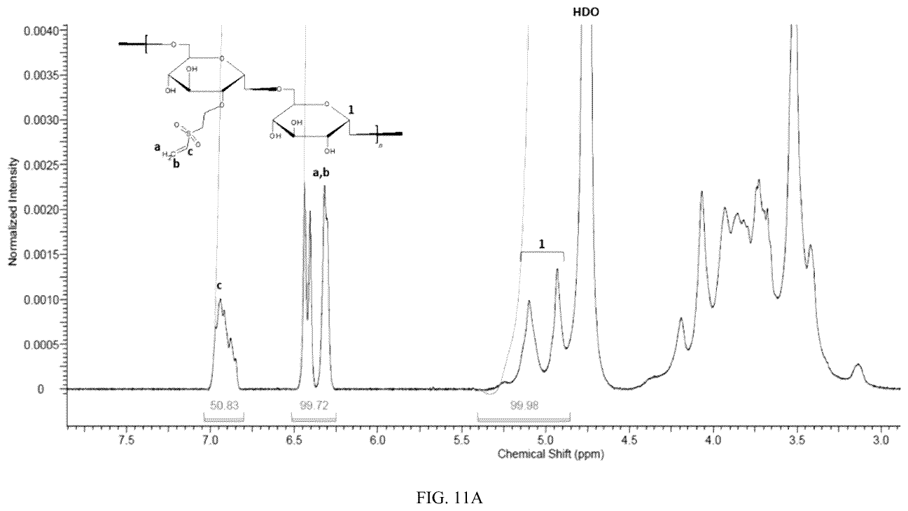

Dextran (DX, 40 kDa) was modified with pendant VS groups as previously reported [18]. Briefly, DX was dissolved at 5% wt/V in 0.02M NaOH solution. DVS (MW=118.16) was added to the DX solution at 1.2:1 molar ratio of DVS to OH on DX with stir mixing under ambient temperature. 0.02M of HCl was added to stop the reaction. The reaction time was carefully controlled to yield VS modified DX (DX-VS) of various degree of modification (DM). The relationship between reaction time and DM was demonstrated in FIG. 10. The polymer was purified with dialysis (Spectra/Por cellulose membrane, 7 kDa MWCO, Spectrum) against ddH₂O for 5 days, and then lyophilized. The DM of VS groups was estimated with 1H NMR spectroscopy (300 MHz, D₂O, residual internal HDO δ 4.75 as reference). The relative number of vinyl protons: δ 6.27-6.44 (q, 2H, ═CH₂), δ 6.82-6.97 (m, 1H, —CH═) were calculated as VS per pyranose unit of DX: δ 4.87-5.29 (m, 1H, C1).

### PCDX Preparation.

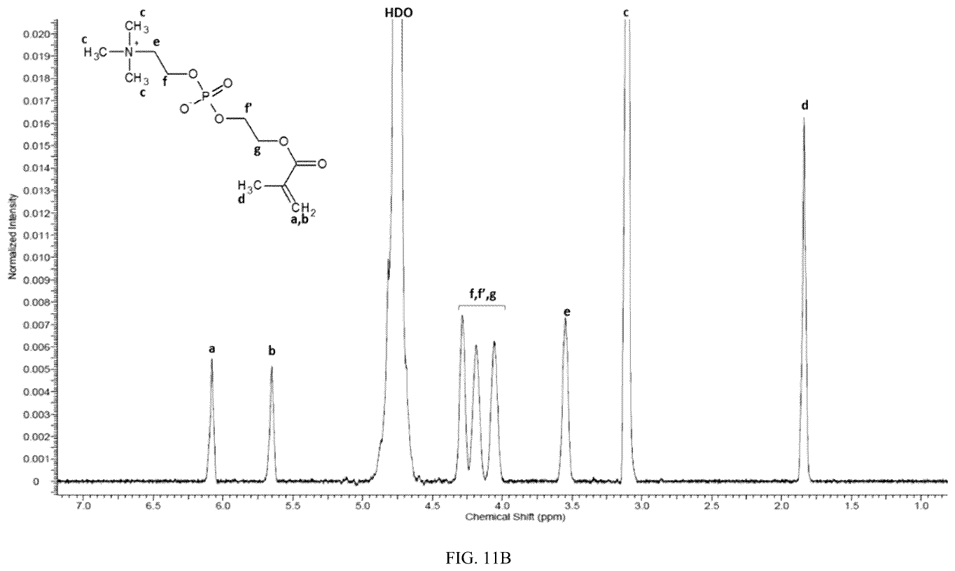

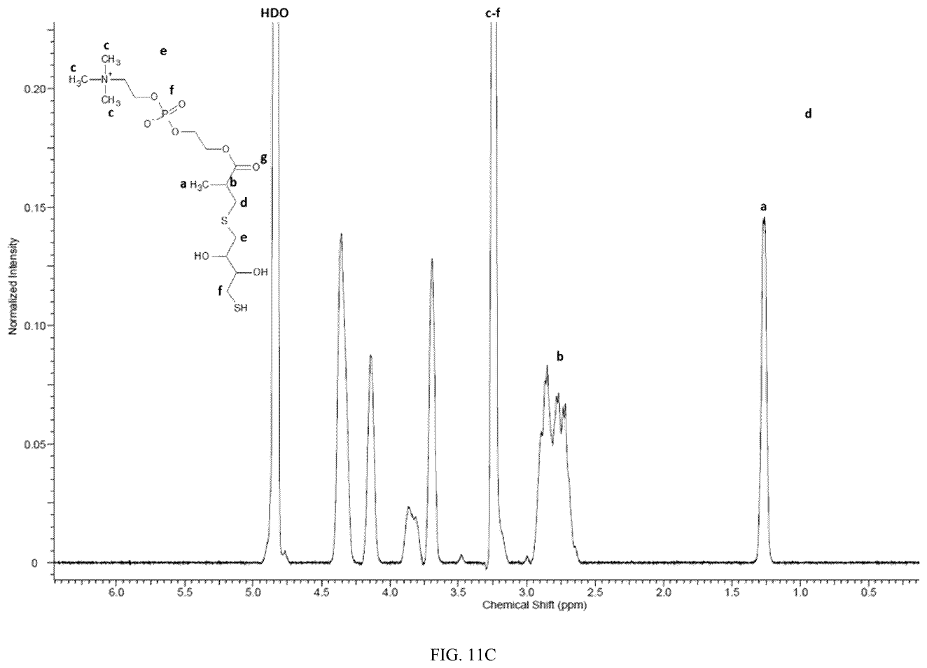

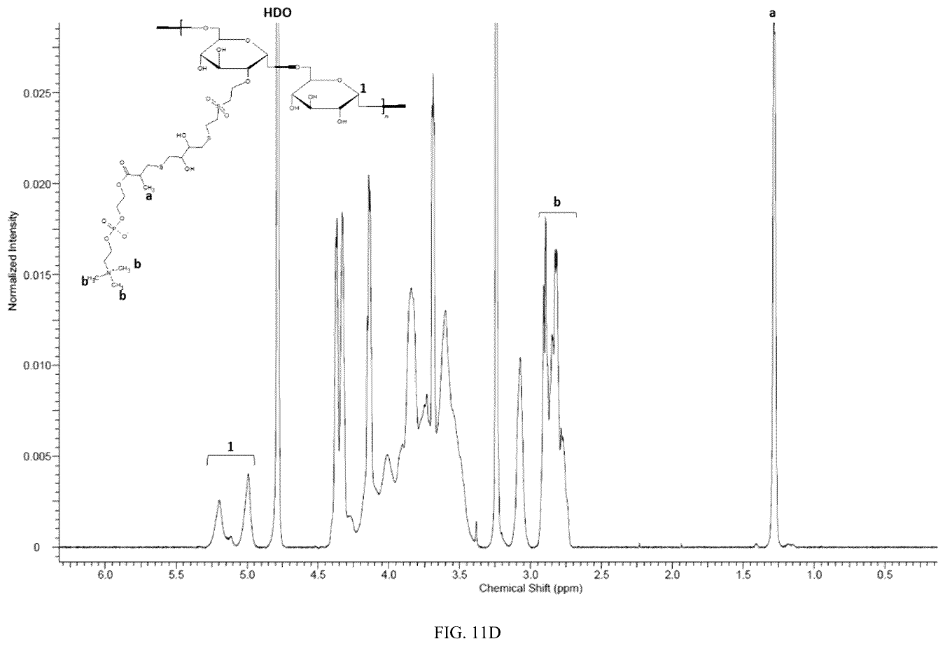

MPC (MW=295.27) was thiolated by reacting MPC with DTT at a molar ratio of 1:6. Briefly, 0.033M MPC was dissolved in PBS (pH 7.5), and 0.2M DTT was added. The reaction was stirred overnight under ambient temperature. The thiolated product MPC-SH was precipitated with acetone. After the evaporation of acetone, the product was dissolved in ddH₂O, lyophilized and stored in −20° C. PCDX was synthesized by conjugating MPC-SH to the DX-VS solution at a molar ratio of 3 to 1. DX-VS of various DM was prepared in PBS at 5% wt/V and subjected to nitrogen purging. A corresponding amount of MPC-SH was weighted, dissolved in ddH₂O and added to the DX-VS solution. The pH of reaction mixture was adjusted to pH 7.5 and reacted for overnight under ambient temperature with stir mixing and nitrogen mixing. The complete consumption of -SH in the reaction was monitored with Ellman's assay according to the manufacturer's instructions. The reaction was stopped by adding 0.1M HCl, and the polymer was purified with dialysis for 5 days against ddH₂O and lyophilized. The ¹H-NMR spectroscopy of DXVS, MPC, MPC-SH and resultant product PCDX was reported in FIG. 11.

### OAc Precursors Preparation.

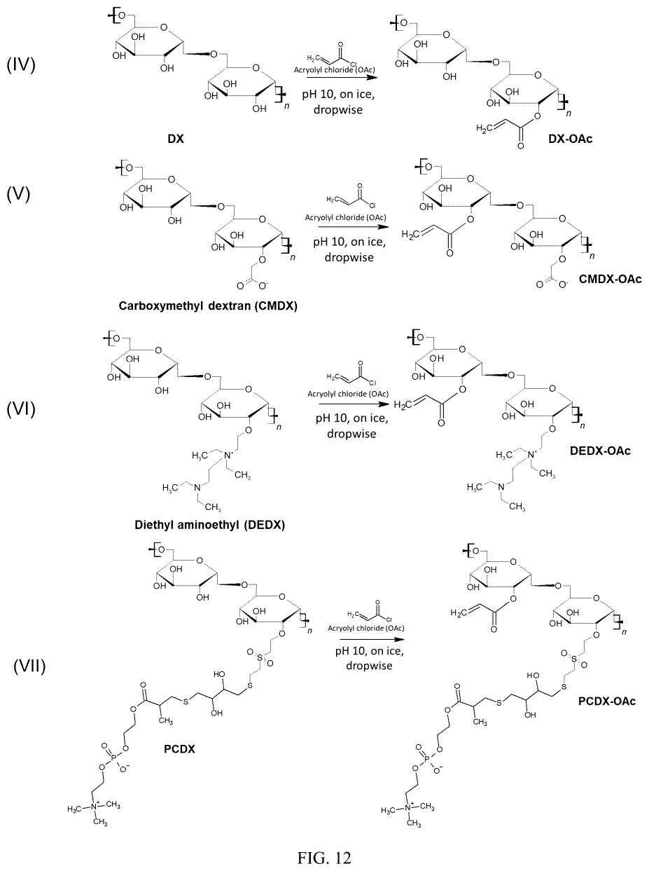

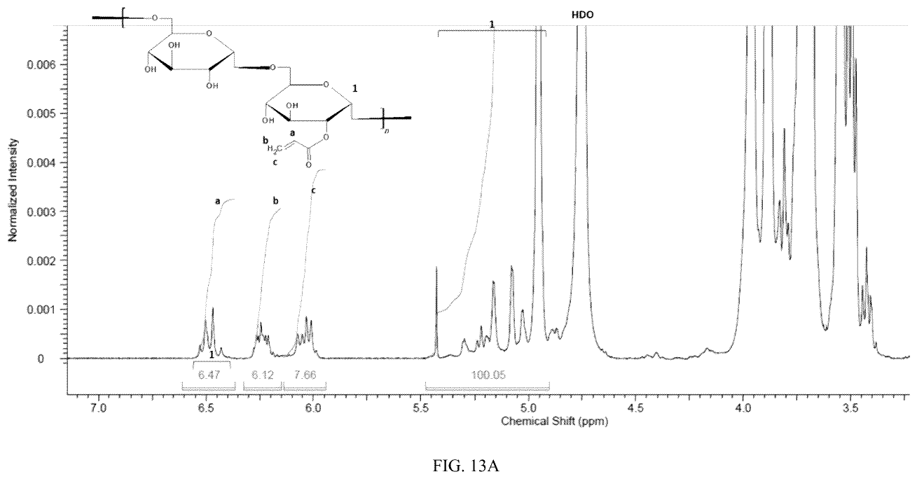

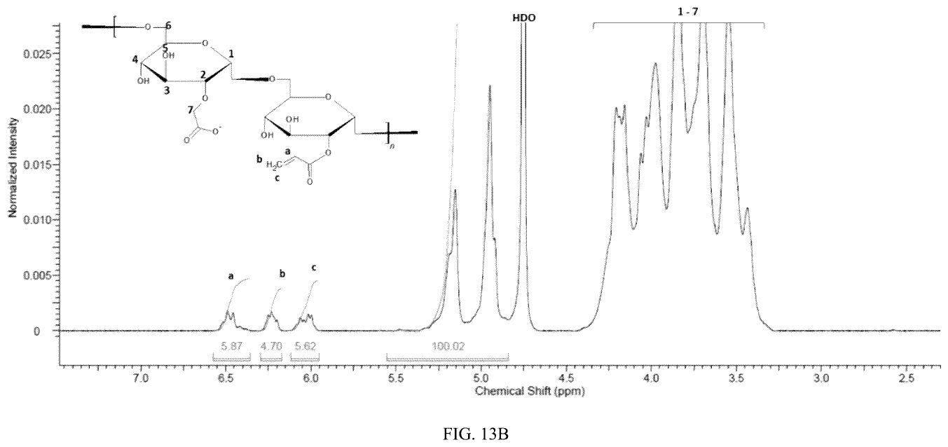

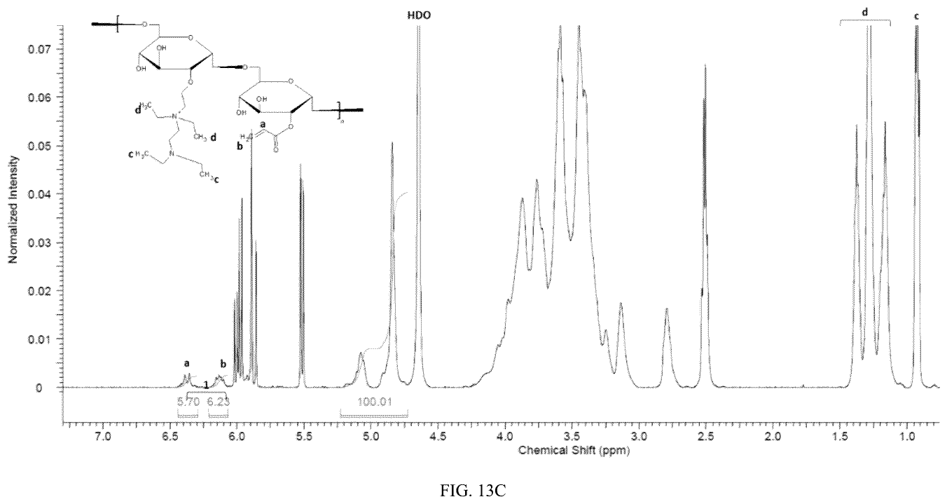

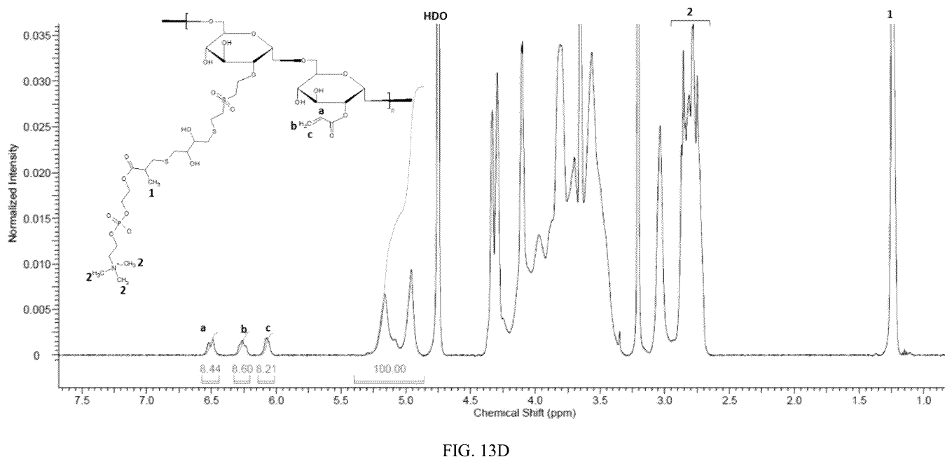

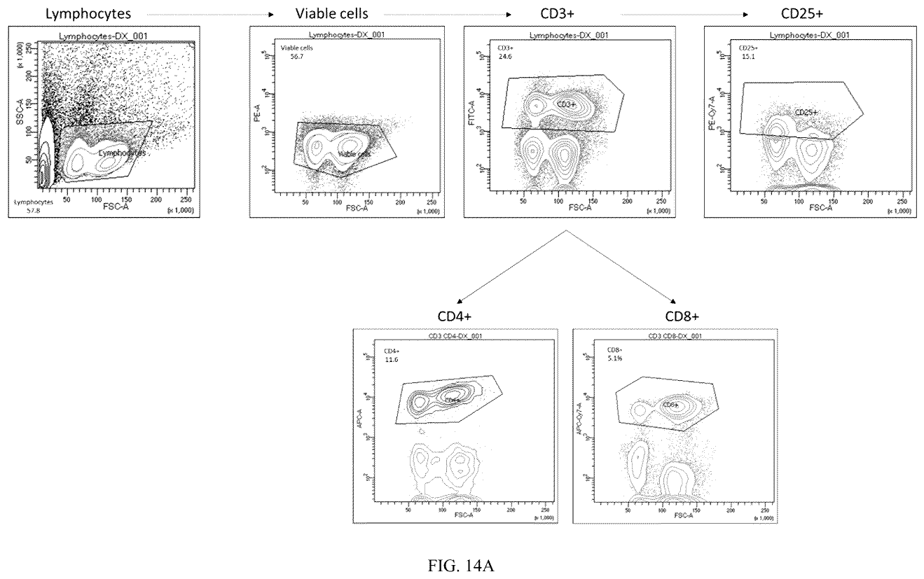

DX and derivatives were functionalized with pendant acrylate groups as previously reported [19]. The synthesis scheme was summarized in FIG. 12. Briefly, DX, PCDX, CMDX or DEDX was dissolved in ddH₂O at 5% wt/V in ice bath. 5M NaOH was added to adjust pH to 10. Acryloyl chloride (MW=90.51) was added to the solution dropwise with a 0.5:1 molar ratio of acryloyl chloride to OH on DX or derivatives. The reaction pH was maintained at 9 to 10 with drop-wise addition of 5M NaOH, stir mixing in ice-bath for 1 hour. The product-OAc was purified with dialysis for 5 days against ddH₂O and lyophilized. The DM of OAc groups was estimated with ¹H NMR spectroscopy (300 MHZ, D₂O, residual internal HDO δ 4.75 as reference). The relative number of vinyl protons: δ 5.90-6.57 (d, 3H, —CH═CH₂) were calculated as -OAc per pyranose unit of DX: δ 4.87-5.29 (m, 1H, C1). The ¹H-NMR spectroscopy of DX-OAc, CMDX-OAc, DEDX-OAc and PCDX-OAc was reported in FIG. 13. In addition, the average DMs of -OAc on the hydrogel precursors estimated from ¹H-NMR spectra were summarized in Table 1.

TABLE 1
Degree of modifications (DM) of -OAc on hydrogel precursors,
estimated from ¹H-NMR spectra

| Hydrogel precursors | -OAc DM(%) |
|---|---|
| DX-OAc | 6.75 |
| CMDX-OAc | 5.43 |
| DEDX-OAc | 6.05 |
| PCDX-OAc | 8.42 |

### Preparation of MPs and OVA-Laden MPs, Measurement of Mechanical Strength

MPs were prepared using droplet microfluidic method according to protocol previously reported with modifications [20]. Briefly, -OAc polymer at 30% wt/V and 0.5M DTT were premixed at 1:1 molar ratio of -OAc to SH. Subsequently, the mixture (pH 6.5) was supplied from one inlet in a microfluidic chip. The light mineral oil with 3% V/V Span80 was used as continuous phase. A pressure controller (OB1 MK3+, Elveflow, France) was used to control the inlets. The droplets were visualized under microscope (Nikon Eclipse Ti-U, Japan). All droplets were collected at collection outlet in a cassette and allowed complete gelation overnight at ambient temperature. The MPs were collected via centrifugation at 3000 r.c.f. for 30 s, and supernatant oil phase was discarded. The mixture was washed in n-heptane and isopropanol to replace the solvent and remove surfactants. Finally, the MPs were collected via centrifugation and resuspended in ethanol for disinfection and storage at room temperature. Prior to characterizations, MPs were collected via centrifugation, ethanol evaporation and resuspension in buffers, such as water or PBS for subsequent cell and animal treatments.

To determine the mechanical strength, the hydrogels were prepared into circular disk with 8.0 mm diameter and 1.0 mm thickness in a mold, as reported previously [13]. Briefly, —OAc precursors were dissolved at 30% wt/V in phosphate buffer and pH was adjusted to pH 6.5. DTT was dissolved at 0.5 mM in ddH₂O, then added at a molar ratio of 0.5 to the -OAc on the modified DX precursors. After thorough mixing, the solutions were transferred to each mold for overnight gelation at 37° C. in a humidified box. The resultant gel disks were mounted on an 8 mm parallel plate fixture. Strain and frequency sweep tests were performed with ARES-G2 (TA Instruments) according to previous protocol [13]. The storage modulus G′ and loss modulus G″ were measured for each hydrogel sample.

### Size and Zeta Potential Measurement

MP sizes were measured by fluorescence microscope (Nikon Eclipse Ti2E widefield microscope) and ImageJ software. A complete z-stack was performed for at least 100 particles to determine the maximum diameters by ImageJ. For the measurement of zeta potential, MPs were resuspended in ddH₂o and adjusted to pH 5, 7, and 9 with 0.01M NaOH and HCl, measured with ZetaPLUS at ambient temperature, and the number of runs per measurement was 5 (Brookhaven Instrument Corporation, US).

### Hydrogel Scaffolds and MPs Preparations for In Vitro and In Vivo FBR Assays

For in vitro assay, the hydrogel scaffolds were pre-formed in 48-well cell culture plates at a volume of 100 μL per well. Briefly, -OAc precursors of CMDX, DEDX, DX, and PCDX were mixed with DTT at -OAc:SH ratio of 1:1 in PB of pH 6.4 to give polymer concentration of 30% wt/V. Prior to gelation, all solutions were filtered with 0.22 μm filter membrane and prepared in sterile conditions to minimize contamination. Subsequently, the hydrogel scaffolds were illuminated with UV radiation for 15 minutes, washed with sterilized PBS three times, and equilibrated with culture mediums for 1 hour. Then, NIH 3T3, RAW264.7 and JAWSII were seeded at 10⁵ cells per 400 μL culture medium on the pre-formed hydrogel scaffolds or tissue culture plate (TCP). On day 3, the cells were stained with LIVE/DEAD viability/cytotoxicity dyes according to the manufacturer's protocols and imaged under fluorescence microscopes. For in vitro cell adsorption assay, the cultures were rinsed with warm PBS three times and imaged again under fluorescence microscope for the number of cells retained on the culture surface.

For in vitro protein adsorption assay, hydrogel scaffolds were pre-formed in 96-well plates and equilibrated in PBS overnight. 0.1 mg/mL fluorescently labelled fibrinogen were incubated on the pre-formed hydrogels and TCP for 12 hours at 37° C. The hydrogels and TCP were washed with PBS five times and subjected to fluorescence measurement with Varioskan™ LUX multimode microplate reader at ex/em 490/520. In addition, the hydrogel scaffolds were also incubated with PBS and measured at the same conditions as blank to determine the hydrogel scattering for noise subtraction. The percentage of protein adsorption were computed by dividing the remaining amount of protein in well over total amount of protein added.

For the preparation of injectable hydrogel scaffolds for in vivo FBR assay, sterile -OAc precursors of CMDX, DEDX, DX, and PCDX were mixed with DTT at -OAc:SH ratio of 1:1 in the syringe on ice bath. Polymer solutions were prepared at 30% wt/V in 150 μL PBS. The solutions were injected subcutaneously at the dorsal region via 27-G needle to allow in situs crosslinking at 37° C. The skin conditions at the injection sites were monitored for 7 days, subsequently the animals were sacrificed, and the hydrogel scaffolds were excised to examine for fibrotic tissue formation and angiogenesis.

### F-OVA Release Profile

Hydrogels encapsulating F-OVA were prepared based on protocol reported previously with some modifications [19]. RAW264.7 and JAWSII were seeded at 10⁵ cells per 400 μL culture medium on the pre-formed hydrogel scaffolds or tissue culture plate (TCP). On day 3, the cells were stained with LIVE/DEAD viability/cytotoxicity dyes according to the manufacturer's protocols and imaged under fluorescence microscopes. For in vitro cell adsorption assay, the cultures were rinsed with warm PBS three times and imaged again under fluorescence microscope for the number of cells retained on the culture surface.

For in vitro protein adsorption assay, hydrogel scaffolds were pre-formed in 96-well plates and equilibrated in PBS overnight. 0.1 mg/mL fluorescently labelled fibrinogen were incubated on the pre-formed hydrogels and TCP for 12 hours at 37° C. The hydrogels and TCP were washed with PBS five times and subjected to fluorescence measurement with Varioskan™ LUX multimode microplate reader at ex/em 490/520. In addition, the hydrogel scaffolds were also incubated with PBS and measured at the same conditions as blank to determine the hydrogel scattering for noise subtraction. The percentage of protein adsorption were computed by dividing the remaining amount of protein in well over total amount of protein added.

For the preparation of injectable hydrogel scaffolds for in vivo FBR assay, sterile -OAc precursors of CMDX, DEDX, DX, and PCDX were mixed with DTT at -OAc:SH ratio of 1:1 in the syringe on ice bath. Polymer solutions were prepared at 30% wt/V in 150 μL PBS. The solutions were injected subcutaneously at the dorsal region via 27-G needle to allow in situ crosslinking at 37° C. The skin conditions at the injection sites were monitored for 7 days, subsequently the animals were sacrificed, and the hydrogel scaffolds were excised to examine for fibrotic tissue formation and angiogenesis.

### F-OVA Release Profile

Hydrogels encapsulating F-OVA were prepared based on protocol reported previously with some modifications [19]. Briefly, the protein was first dissolved in -OAc functionalized DX polymers (40% wt/V in phosphate buffer) and adjusted to pH 6.5. Hydrogels were formed by crosslinking DTT with -OAc, the ratio of SH to OAc was maintained at 1 to minimize undesired -OAc to protein binding. After thorough mixing, the solution of 30 μL were pipetted on a hydrophobic surface to form hemispherical droplet, and subsequently incubated in a humid chamber at 37° C. overnight to ensure gelation. To determine the protein release profile, the hydrogel hemispheres were incubated in 1 mL PBS as releasing buffer at 37° C. PBS was collected and replenished at each time point, and the concentration of F-OVA was measured by spectrophotometry at ex/em 490/520 nm using black 96-well plate in Varioskan™ LUX multimode microplate reader. Graphs of cumulative release percentage of F-OVA versus time were plotted for DX, CMDX, DEDX and PCDX hydrogels.

JAWSII Interactions with MPs

To monitor JAWSII interactions with MPs, confocal fluorescence cell imaging was performed. MPs were chemically conjugated with FITC for visualization. Briefly, VS precursors of CMDX, DEDX, DX, and PCDX were prepared at 30% DM and conjugated with cysteine-FITC at molar ratio VS:SH of 3:1. The remaining VS was fully reacted with DTT at VS:SH molar ratio of 1:1 to form hydrogel networks in the droplet synthesis microfluidics method. Unlike the -OAc formulations, MPs prepared in this method were not susceptible to hydrolytic degradation and were used to monitor cell-gel interactions. JAWSII were seeded at 10⁵ cells in 1 mL medium in confocal dish and 100 μL OVA or MPs loaded with OVA in PBS were added. After 24 h treatment, cells were incubated with LysoTracker™ Red DND-99 and Hoechst 33258 according to the manufacturer's protocols, and then fixed with 1% paraformaldehyde. Fluorescent images were recorded with Leica SP8 confocal microscope.

### Quantitative Analysis of Antigen Uptake by JAWSII

For antigen uptake assay, JAWSII was seeded at 10⁵ cells per mL in 600 μL medium in 24-well dish. Subsequently, the cells were treated with 50 μL F-OVA or MPs loaded with F-OVA at 37° C. and 4° C., to determine the background level of uptake assay. The treatment concentration of OVA was kept consistent at 20 μg/mL, MPs polymer concentration is 40 mg/mL in PBS. Cells were treated for 24 h and at the end of incubation, cells were harvested, washed with chilled PBS two times and resuspended in PBS for flow cytometry using BD FACS Aria III Cell Analyzer. Mean fluorescence intensity (MFI) and F-OVA⁺ cell population % were used to estimate OVA uptake level.

### MP Immune Phenotyping: Surface Markers Expression and Cytokine Generation

To evaluate the maturation of JAWSII, cells were cultured seeded at 10⁵ cells per mL in 600 μL culture medium in 24-well plate. The next day, cells were treated with 50 μL OVA, MPs loaded with OVA for 24 h. The treatment concentration of OVA was kept consistent at 20 μg/mL, MPs polymer concentration is 30 mg/mL in PBS. Subsequently, cells were labelled with fluorescent-probed antibodies against, CD86, CD40, CD206, CCR7, I-Aκ(Aβκ), and H-2K^{b} bound to SIINFEKL, according to the manufacturer's instructions. All markers were measured by flow cytometry on BD FACS Aria III Cell Analyzer. Samples were recorded at 5000 to 10000 events per run and analyzed along with respective isotype and unstained controls. Data was analyzed with BD FACSDiva™ software, and the level of surface marker expression was determined by MFI and was presented as the ratio of change relative to non-treated cells.

For cytokine detection, the supernatant of cell culture medium was collected after treatments. TNF-α, IL-6, and IL-1β were measured with ELISA kits according to the manufacturer's protocols. The effective endotoxin concentration released from MPs sampled from PBS in MPs suspension was measured using chromogenic LAL endotoxin assay according to the manufacturer's protocol (ToxinSensor™, Genscript, US) (Table 2).

TABLE 2
The average endotoxin level released from hydrogel
washed with PBS after 24 hours incubation.

| Hydrogels | Average Endotoxin Level (EU/mL) |
|---|---|
| CM | 0.165 ± 0.045 |
| DE | 0.109 ± 0.015 |
| DX | 0.125 ± 0.004 |
| PC | 0.197 ± 0.002 |
| PBS | 0.0128 ± 0.004 |

### In Vivo Recruitment and Activation of Dendritic Cells and Macrophages by MPs

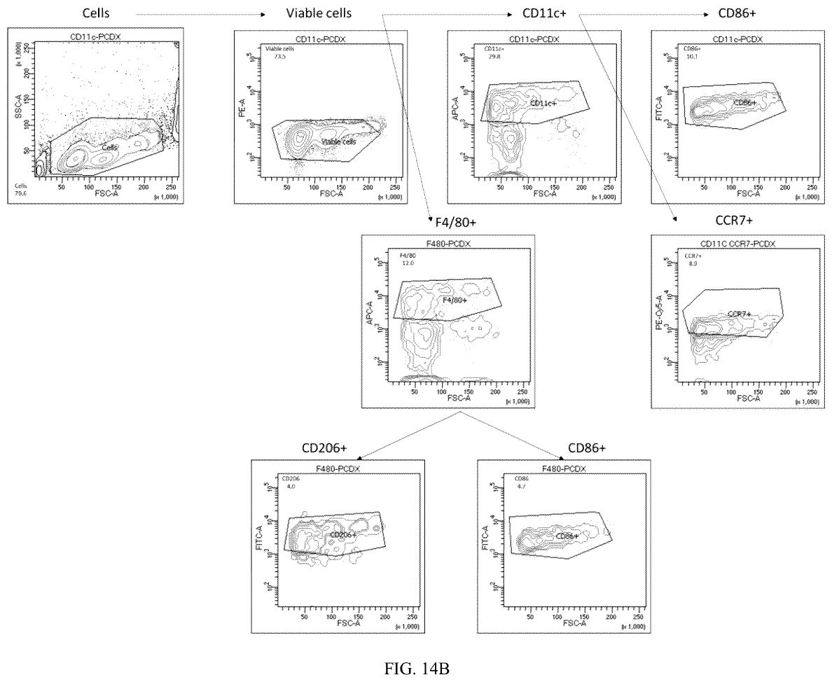

Subcutaneous areas containing MPs were excised for flow cytometric analysis of recruited DCs and macrophages. Samples were collected at day 7 after MP injection. The sample blocks containing MPs were digested with collagenases and dextranase. Subsequently, the single-cell suspensions generated were blocked with 1% NGS and stained with CD11c, F4/80 for DC and macrophage populations respectively, as well as CD86, CCR7 for activation and lymph node homing markers. The stained cells were analyzed with BD FACS Aria III Cell Analyzer. Samples were recorded at 5000 to 10000 events per run and analyzed along with respective isotype and unstained controls. Data was analyzed with BD FACSDiva™ software, and the populations of CD11c⁺, CD86^{hi}, CD11c⁺, CCR7⁺, F4/80⁺, CD86^{hi}, and F4/80⁺, CD206^{hi} were gated and compared with all treatment groups. The gating strategy was summarized in FIG. 14.

### OVA Biodistribution Analysis In Vivo

OVA was conjugated with infrared dye, IRDye® 800CW NHS ester according to the manufacturer's instructions. Mice were injected subcutaneously at the dorsal region with 150 μL OVA-IR or OVA-IR loaded in MPs in PBS. The dosage of OVA-IR was kept consistent in all treatment groups (350 μg per animal), same for MPs polymer concentration (10 mg per animal). The average animal weight was 22 to 28 g. Sham group was injected with same volume of PBS and the organs were used as blank. At determined time, the animal was sacrificed to harvest organs, skin at injection site, inguinal lymph nodes, spleen, kidneys, liver, and lungs. The hair of C57BL\6 mice in injection site was removed to reduce autofluorescence. Fluorescence imaging was performed on the organs of each group to observe the accumulation of OVA-IR using Odyssey Infrared Imaging System (LI-COR Biosciences, US) 800 channel laser source. The results were analyzed using Image Studio Lite Version 5.2 software.

### MP-Induced T-Cell Responses

On day 7 post MPs/OVA injections, the spleens were harvested and splenocytes were collected according to the isolation protocol. The isolated single cell suspensions were stained with appropriate fluorescent-labelled antibodies: anti-mouse CD3, anti-mouse CD4 and anti-mouse CD8b. The cell surface markers were analyzed by flow cytometry BD FACS Aria III Cell Analyzer. Samples were recorded at 10000 events per run and analyzed along with respective isotype and unstained controls. Data was analyzed with BD FACSDiva™ software, and the populations of CD3⁺, CD3⁺ CD4⁺, CD3⁺ CD8⁺, and CD3⁺ CD25⁺ were gated and the gated population percentage were compared with all treatment groups. The gating strategy was summarized in FIG. 14.

### OVA-Specific Antibody Responses

Serum samples were collected via cardiac puncture at day 7, 14 and 28 for the antibody detection. The levels of anti-OVA IgG1 and IgG2a were measured with ELISA according to the manufacturer's instructions. Sera were prepared at 1:500 dilutions and the absorbance at 450 nm was measured.

### Statistical Analysis

Data are represented as mean±standard deviation (s.d.). For statistical significance analysis, data were analyzed with an ordinary one-way Analysis of Variance (ANOVA) in GraphPad Prism 7 software to compare each treatment group with the corresponding control groups.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Bulk Hydrogels Preparations and Physical Characterizations

Dextran (DX) was chosen as the depot carrier for its versatility and immunomodulating propensity. DX has been utilized as blood expander and stabilizer in cell therapy infusion products. Furthermore, the hydroxyl (—OH) groups in DX backbones can be activated for further chemical modifications. In terms of immunomodulation, DX is a ligand to C-type lectins (CLRs) and activates immune cells such as DCs, macrophages, and monocytes [21]. DX-adsorbed nanoparticles can promote higher antigen phagocytosis by DCs than non-modified form. Moreover, DCs cultured on DX hydrogel surface also exhibit stronger maturation profile than cells on tissue culture plate [13, 22]. On the other hand, zwitterionic polymers or coatings have been widely adopted to bypass FBR of medical implants in vivo. Phosphorylcholine (PC) is of particular interest as it is analogous to the phospholipid plasma membrane that renders the cells resistant to non-specific protein adhesion and cell-cell interactions. Also, devices fabricated with PC-based polymers exhibit excellent biocompatibility and anti-fouling propensity. PC-based polymers can suppress the adhesion of serum proteins, inflammatory proteins as well as macrophage and other immune cells on the medical implants in vivo and in vitro [23, 24].

Besides the biomaterials, the dosage forms of antigen delivery at the subcutaneous region are also crucial in regulating the innate immune outcome in favor of vaccination [25, 26]. It has been demonstrated that carriers of micro and meso size range are preferred in long-term vaccine development to achieve localized and sustained immune modulation [27-29]. We compared two injectable forms of antigen depot, namely bulk hydrogel or a collective of hydrogel microparticles (MPs) and investigated the extent of their impact on the interactions between the resident immune cells and biomaterials.

We hypothesized that hydrogels made of both immunogenic DX and zwitterionic PC could engage DCs for cell maturation while evading FBR. We chemically grafted PC on DX-based hydrogel precursors (PCDX) and developed two injectable forms—bulk hydrogel scaffold and MPs—for controlled antigen delivery. To validate the role of zwitterionic PC in circumventing FBR, we compared the results with various charge derivatives of DX polymer, including the neutral native dextran (DX), anionic carboxymethyl (CMDX) and cationic diethyl aminoethyl (DEDX) dextran in constructing the hydrogels for antigen delivery and immunomodulation in vitro and in vivo.

In comparison to other charge derivatives, PCDX could effectively evade FBR as anionic CMDX, but the latter was susceptible to rapid swelling and release of cargoes and triggered less robust adaptive immune responses. On the other hand, DX, and DEDX bulk hydrogels and MPs induced prominent FBR, supported with macrophage M1 and M2 polarizations, adsorption of fibrinogen and fibroblasts, resulting in isolation of antigen depots from host tissue environment. PCDX stood out as promising immunogenic depots for sustained antigen delivery.

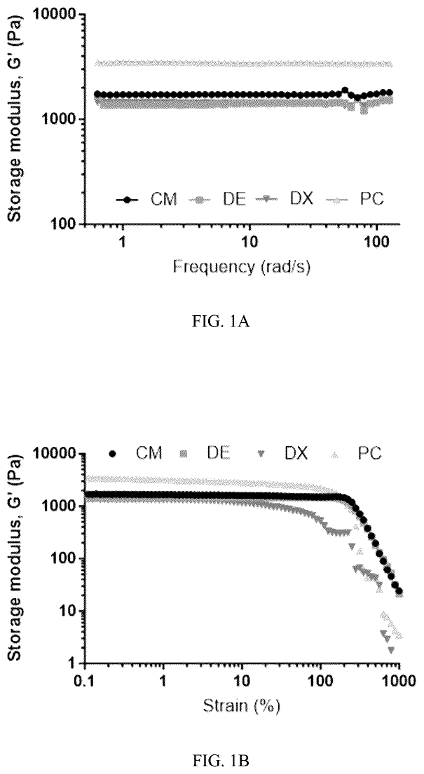

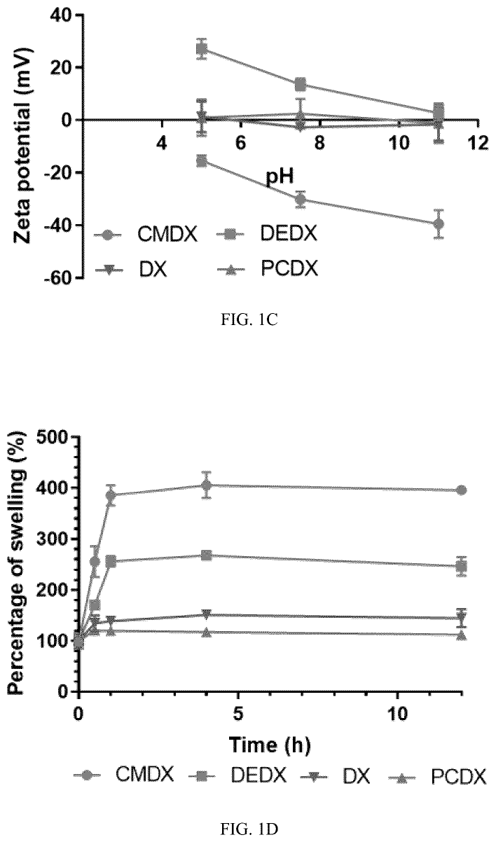

We successfully synthesized zwitterionic DX, PCDX by grafting thiolated methacryloyloxyethyl phosphorylcholine (MPC) on DX backbones that had been functionalized with vinyl sulfone groups (VS) via Michael addition, as shown in FIG. 9 and FIG. 11. In addition, we also introduced “clickable” moieties acryloyl (-OAc) groups onto DX and charged derivatives, forming PCDX-OAc, CMDX-OAc, and DEDX-OAc as summarized in FIG. 12. The structures were confirmed by ¹H NMR as reported in FIG. 13. The -OAc activated precursors allowed the polysaccharides to “click” and crosslink with -SH in DTT to form the hydrogel networks. These hydrogels exhibited elasticity dominating behavior as shown in the frequency and strain sweeps (FIGS. 1A-1B). The average storage modulus, G′ of resulting DTT-crosslinked DX, CMDX, DEDX, and PCDX hydrogels were 1415.07±460.99 kPa, 1703.13±51.55 kPa, 1522.75±280.55 kPa, and 3480.44±779.74 kPa, respectively. The elasticity of reported hydrogels was similar to that of hypodermis (2 kPa) and adipose tissues (2 to 4 kPa), suggesting extracellular matrix mimicking environment to support cell growth and differentiation. Notably, PCDX demonstrated highest G′ due to the additional electrostatic interactions between the zwitterionic phosphorylcholine groups that strengthen the biopolymers crosslinking.

In terms of surface charge, DX and PCDX, which consisted of equal molar of anionic phosphoryl and cationic quaternary amine moieties, are both neutral and independent of pH change. On the other hand, the carboxyl groups containing CMDX and tertiary amine groups containing DEDX are negative and positively charged respectively at pH 7.5 (FIG. 1C). Additionally, the hydrophilicity of DX backbone allowed influx of aqueous buffer and causing hydrogel to swell in PBS with time, as demonstrated in FIG. 1D. PCDX showed the least rate and size due to the attractive force between the opposite charges; conversely, CMDX and DEDX achieved greater swelling sizes than PCDX and DX due to repulsion between the like charges. The charge decorations affected the mechanical and swelling behaviors of DX hydrogels as expected.

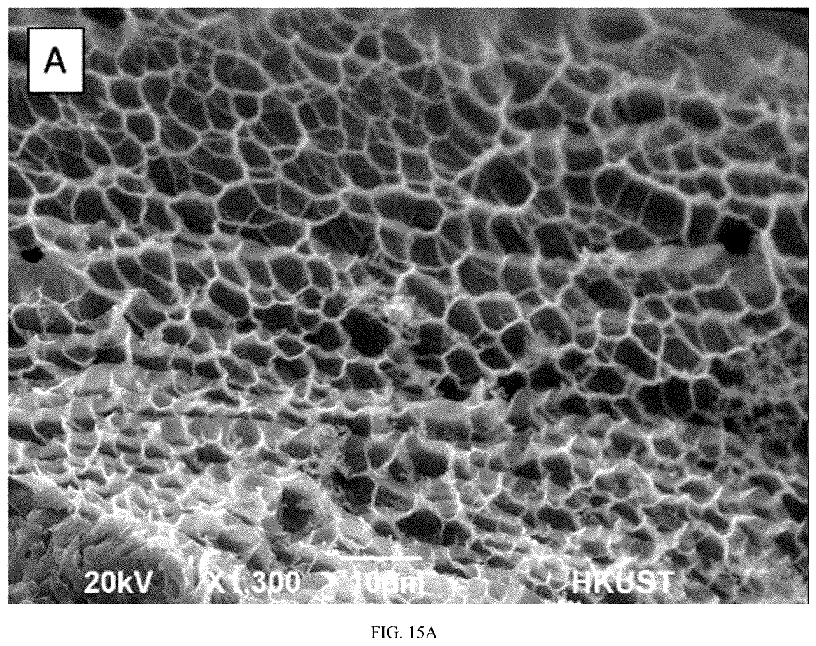

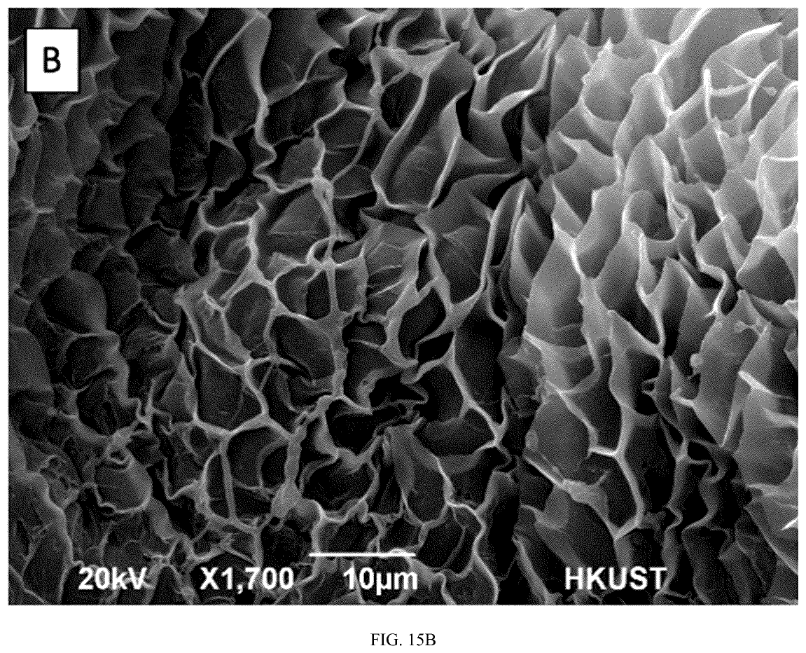

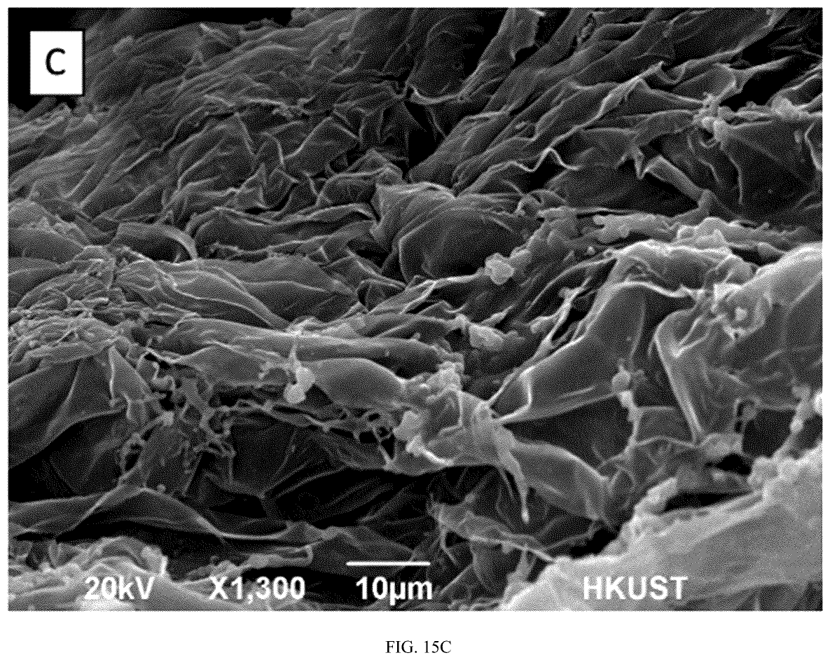

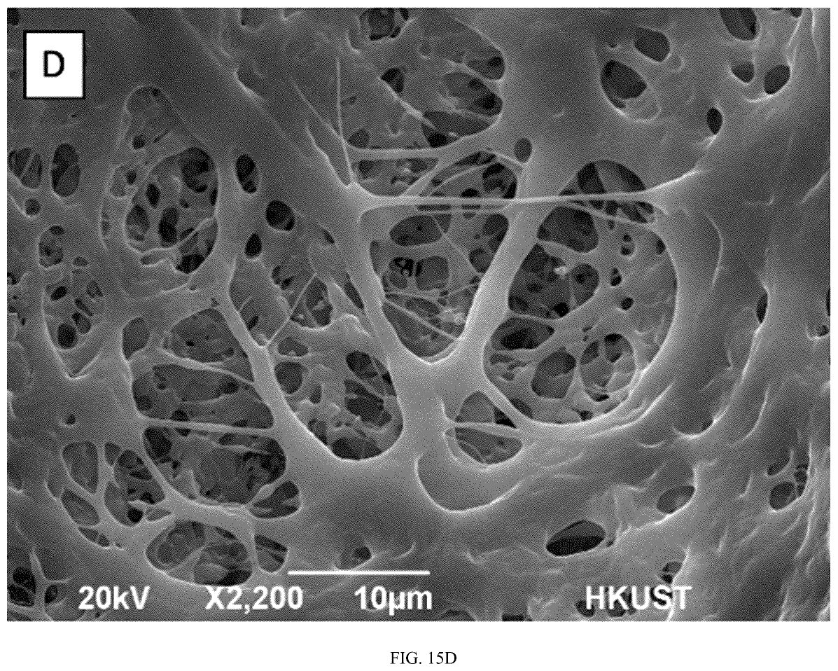

Hydrogel of varying surface charges also displayed different microscopic porous structures as shown in FIG. 15. The scanning electron microscope (SEM) images were obtained from hydrogels after swelling in water and subjected to lyophilization. The results revealed that DX exhibit a porous architecture characterized by a more uniform distribution of pores. In contrast, the CMDX hydrogel displays larger pores and collapsed porous structure, which could be due to more rapid swelling of hydrogel with charge-charge repulsion, and hydrogel compaction with centrifugation prior to SEM characterization. Similarly, DEDX exhibited large pores as a result of rapid hydrogel swelling with cationic charge-charge repulsion. Conversely, PCDX displayed less uniform porous structure. It shows smooth surface, attributable to the zwitterionic properties of the phosphorylcholine modification, which minimizes electron scattering of SEM and results in a smoother surface.

### Example 2—DX-Based Bulk Hydrogels Induced Foreign Body Responses

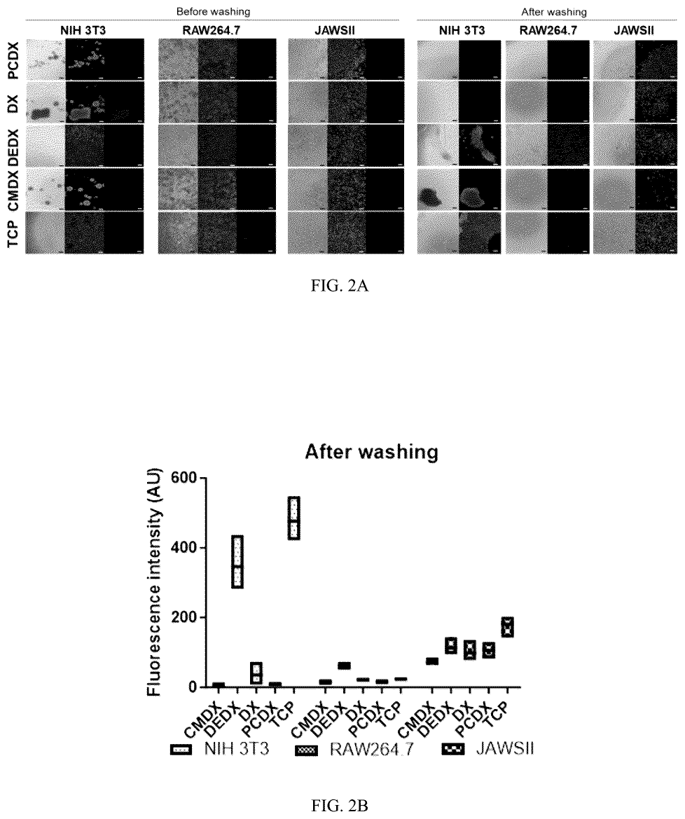

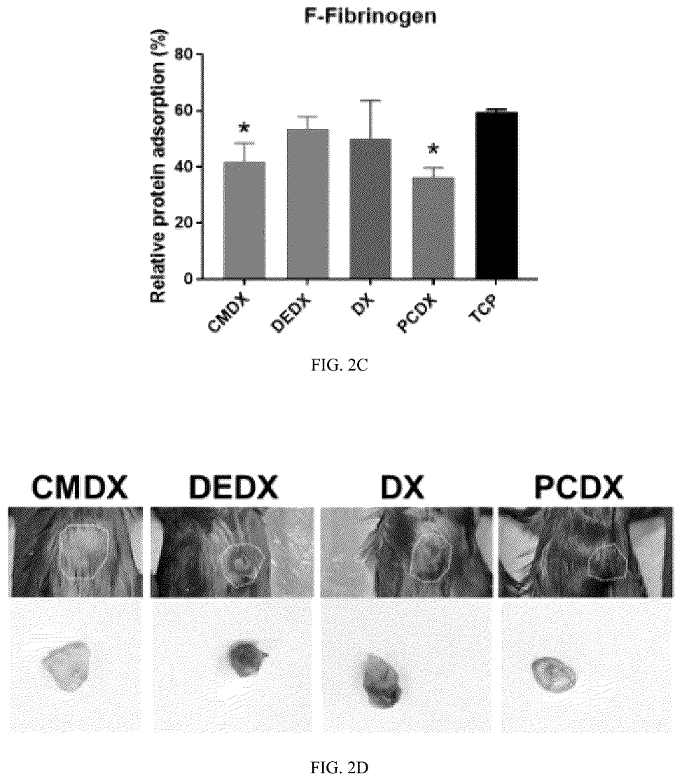

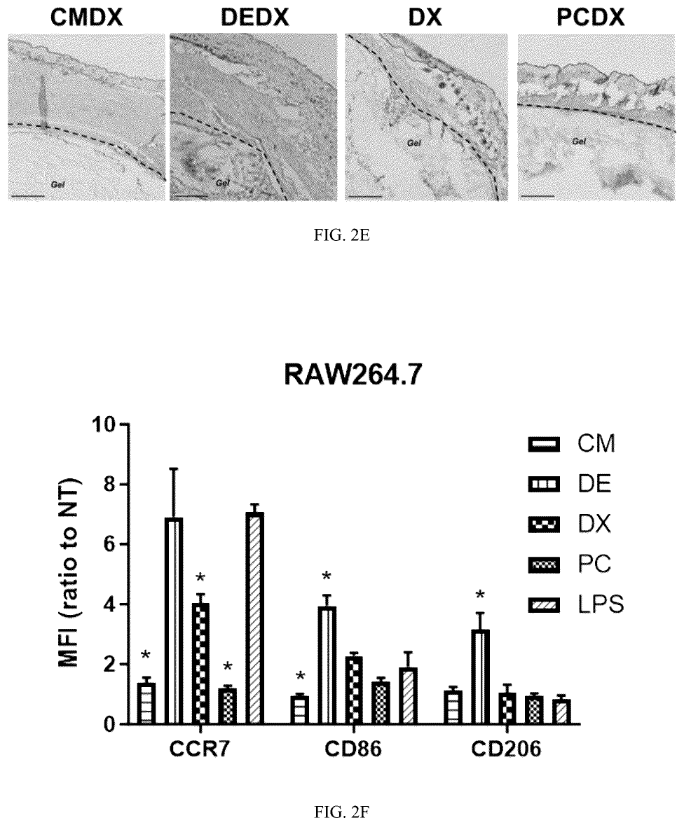

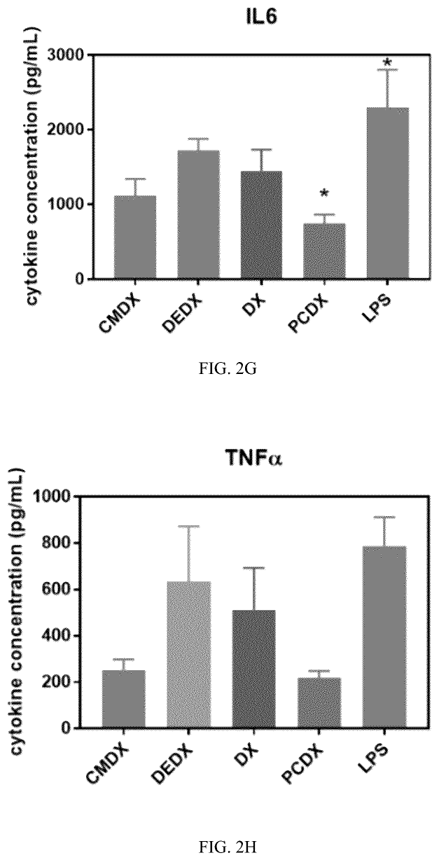

All hydrogels demonstrated good biocompatibility. The cytocompatibility of DX-based hydrogels has been verified with Live/Dead viability assay in FIG. 2A with more than 70% of cells, from NIH 3T3, RAW264.7, and JAWSII exhibit calcein AM activity in vitro. the histocompatibility assessment, 150 μL gel solutions were injected subcutaneously and allowed for in-situ crosslinking at 37° C. body temperature. The experimental mice exhibited normal eating and behavior with no significant weight loss. The skin conditions were monitored continuously for 7 days. In comparison to other hydrogel groups in FIG. 2D, PCDX did not exhibit significant skin rash, swelling and bleeding at the skin area post-injection, suggesting good histocompatibility of PCDX.

The foreign body responses (FBR) were probed in vitro and in vivo by measuring responses of relevant cell lines and tissue fibrosis of hydrogels in immunocompetent C57BL\6 mice. In the in vitro assays, we adopted NIH3T3, representing fibroblasts that adhere to the biomaterial surface and secrete collagenous matrix to isolate biomaterials from the host tissue environment. As shown in FIG. 2A, fibroblasts on CMDX and PCDX formed aggregates and were easily rinsed off with PBS washing, implicating the anti-fouling properties of anionic and zwitterionic surfaces of CMDX and PC respectively in resisting the attachment of anionic cell surface. On the other hand, the numbers of NIH3T3 adhered on DEDX for 24 hours were similar to that of TCP and exhibited similar morphology to that of TCP; nonetheless, the cells attached on DX were lower. After rinsing with warm PBS, the cells remain attached on the culture surface of DEDX and TCP.

We also verified the interaction of PCDX with immune cell lines, RAW264.7 and JAWSII, in comparison to other DX derivatives hydrogels. Macrophages RAW264.7 are suspension cells in inactivated state and become adhered when activated [30]. FIGS. 2A-2B showed significantly lower number of macrophages adhered on PCDX and CMDX as compared to other treatment groups before and after rinsing of hydrogel culture surface with PBS. Moreover, the macrophages on PCDX and CMDX did not exhibit significant M1 or M2 polarizations, in terms of change of M1 markers CCR7, CD86, IL-6, TNF-α as well as M2 marker CD206 (FIGS. 2E-2G). This suggests that PCDX and CMDX effectively resisted the engagement of macrophages, in contrast to DX and DEDX. Notably, the macrophages adhesion on DEDX was the highest amongst the treatment groups. The cells cultured on DEDX also demonstrated significant M1 and M2 polarizations, which are crucial indicators to FBR progression [31-32]. On the other hand, it has been reported that the adhesion of DCs on biomaterials could support cells maturation and immune activation [33]. We observed the number of adherent JAWSII on PCDX was higher than CMDX after rinsing with PBS, indicating certain extent of interactions of the biomaterials with JAWSII. Besides the cell-biomaterial interactions, it has also been well established that the adsorption of fibrinogen on implants could promote FBR and pro-inflammatory responses [34]. The propensity of protein adhesion on hydrogels were probed with fluorescently labelled fibrinogen (340 kDa, pI 5.8). As shown in FIG. 2C, PCDX demonstrated similar anti-fouling propensity as CMDX, of which it could significantly resist the adsorption of F-fibrinogen as compared to TCP, DX, and DEDX.

Next, we also examined FBR in vivo on immunocompetent C57BL\6 mice. The precursors were injected subcutaneously and allowed in-situ crosslinking to form hydrogels at 37° C. as previously reported [35]. We observed the formation of skin ulcers at the injection sites of DEDX treated groups, N=6 starting from day 7 to day 21, suggesting strong chronic pro-inflammatory responses and impairment of the wound healing process. In FIG. 2D, some blood vessels and brownish tissues were formed around the hydrogels. Furthermore in FIG. 2E, the tissue sections with H&E staining also revealed the extent of cell recruitment, tissue fibrosis and angiogenesis. Based on these results, the FBR trend was DEDX>DX>PCDX>CMDX in a descending order. In DEDX and DX, the formation of fibrotic capsules surrounding the injected bulk hydrogels were more extensive than other hydrogel groups resulted in the isolation of biomaterials and the payloads from the host environment. Although CMDX could significantly suppress FBR in vitro and in vivo, the bulk hydrogel underwent swelling by 4 times of original size upon injection, causing significant tissue distortion. To circumvent this problem, we prepared hydrogel microparticles (MPs) of PCDX and compared it with similar injectable forms of CMDX, DX and DEDX in terms of the immunomodulation propensity in vitro and in vivo.

### Example 3—DX-Based Microparticles (MPs) Preparations and Characterizations

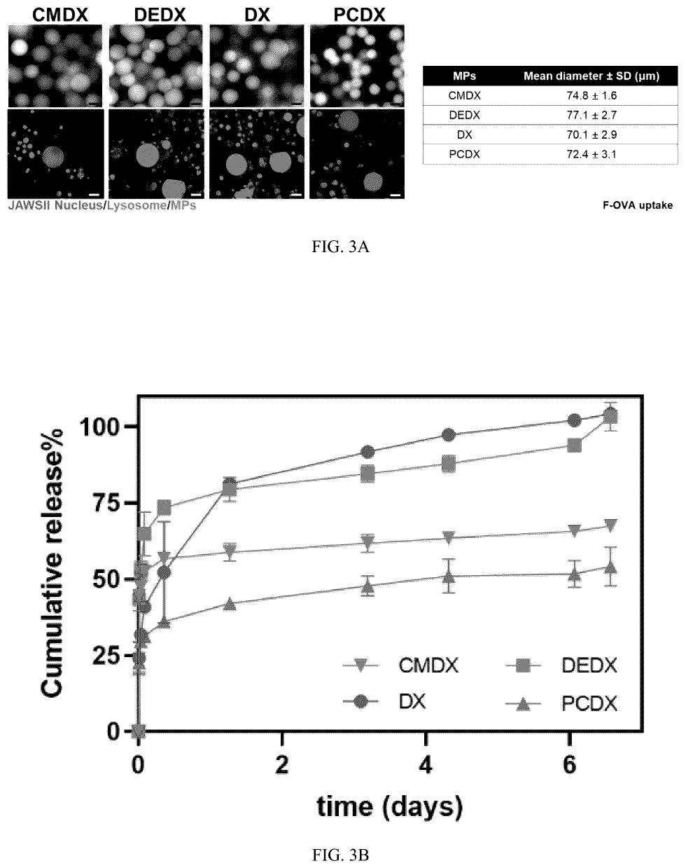

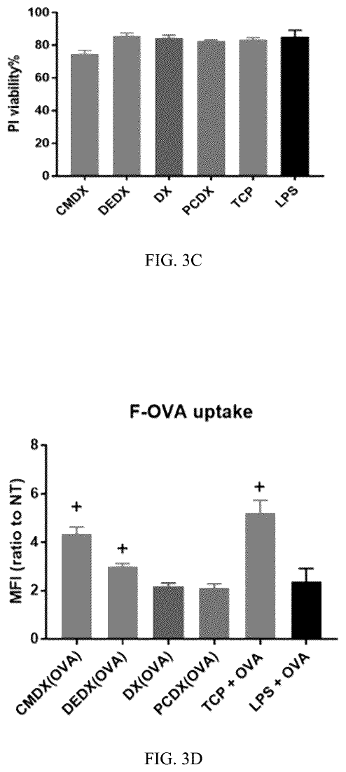

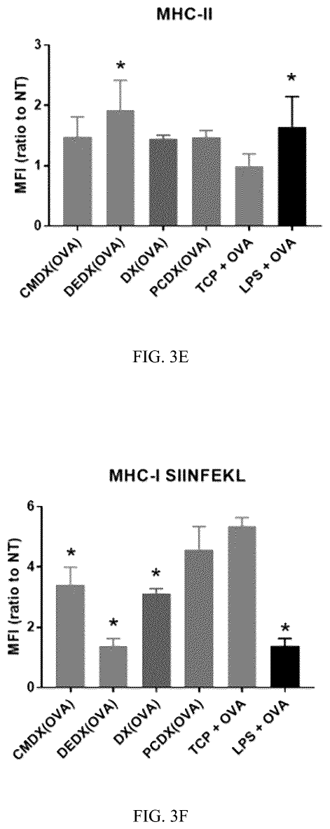

As the bulk hydrogels prepared from DX and derivatives formulations are limited by the greater propensity of FBR, greater hydrogel swelling and lower surface area for target cell interactions, we decided to micronize the hydrogels into microparticles (MPs) to deliver protein-based antigens to the dendritic cells. We successfully prepared MPs, containing ovalbumin (OVA, 45 kDa, pI 4.5) as the model antigen with same formulations as the bulk hydrogels using microfluidics droplet synthesis method as previously reported (FIG. 3A). As summarized in FIG. 3B, the mean diameters are 74.8±1.6 μm, 77.1±2.7 μm, 70.1±2.9 μm and 72.4±3.1 μm for CMDX, DEDX, DX, and PCDX respectively. The diameter range of 70 to 80 μm is preferred in our MPs design to help evading the systemic, phagocytic clearance of MPs and promote local retention of MPs at the subcutaneous region to provide spatially controlled release of antigens to resident DCs [24 to 27]. Our MPs have a diameter similar to titan cryptococcal cells of 50 to 100 μm that can effectively bypass the phagocytosis by immune cells [37].

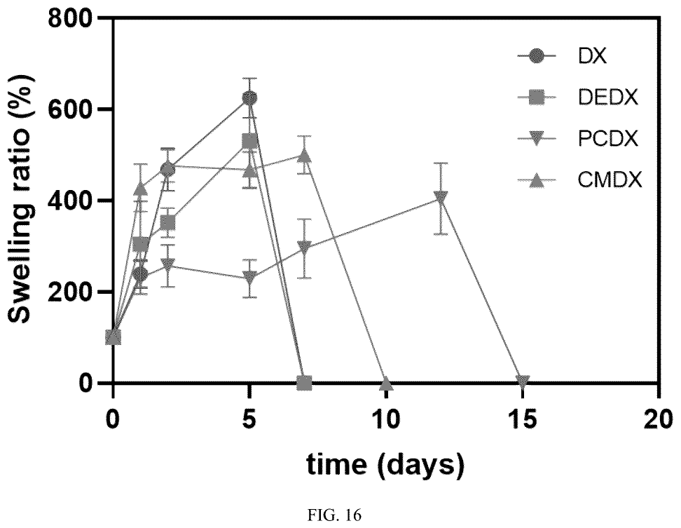

The degradability was also examined with bulk hydrogels prepared with -OAc modified precursors. Based on the hydrogel design, the conjugation of acrylate (-OAc) at the linker region allowed hydrogel degradation via the hydrolysis of ester bonds, as illustrated in FIG. 16. To determine the hydrolytic hydrogel degradation in vitro, the swelling ratio of four DX-based hydrolysable hydrogels were monitored in PBS (pH 7.4) at 37° C. with time. This method has been previously reported [28 to 19]. The hydrogel degradation time is defined as the time needed for complete degradation (Wₜ=0), that is the disappearance of interface between hydrogel and buffer. The swelling ratio result of degradation OAc DX bulk hydrogel formulations is shown in FIG. 16.

In the initial timepoints, DEDX and CMDX experienced significant swelling, which could be due to the charge-charge repulsion of the cationic and anionic polymer backbones of DEDX and CMDX respectively. DEDX continued to swell more rapidly than CMDX. This could be due to the rapid ester degradation of the hydrogel linkers, which were accelerated by the basic DEDX hydrogel microenvironment. By day 7, DEDX and DX experienced complete degradation. Conversely, PCDX and CMDX showed slower degradation than DEDX and DX. The gel-buffer interface began to disappear in 10 days for CMDX and 15 days for PCDX. For the case of CMDX, it was reported that the anionic CM moieties could reduce the hydrolytic rate of the polymer chains [38]. Whereas in PCDX, we speculated that the anionic phosphoryl and cationic choline groups have achieved a net charge balance and resisting the engagement of ions and water molecules in causing ester hydrolysis within the hydrogel microenvironment.

For in vitro biological evaluations, when the MPs were co-cultured with JAWSII dendritic cell line, we observed significantly less cell-gel interactions in PCDX and CMDX groups, which could be due to the anti-fouling surface of MPs and slower degradation rate of polymer chains. Conversely, DEDX and DX groups demonstrated higher cell adhesion on MPs and the distribution of lysosomes surrounding the MPs were higher (FIG. 3A). The rapid internalization of hydrogel products could be due to the rapid degradation rate of DEDX and DX than CMDX and PCDX (FIG. 3B), as well as the chemical entities of the hydrogel carriers that could influence the maturation phenotypes of JAWSII and the resulting antigen uptake and processing pathways.

The release rate of OVA from MPs was measured. As shown in FIG. 3B, OVA was subjected to burst release within 6 hours for all MP groups, with 50 to 70% cumulative release from CMDX, DX and DEDX. Conversely, OVA release from PCDX was significantly lowered to 38% in the initial timepoint. Overall, the release profiles for all hydrogels followed a pseudo first-order release kinetics, with the initial burst release dominated by the diffusion of OVA as hydrogel swells and buffer influx, while subsequent release followed the degradation of hydrogel over time at 37° C. DEDX and CMDX experienced more rapid release initially, which could be due to the rapid swelling of hydrogel and repulsion between the like charged hydrogel backbones and cargoes at pH 7.5 PBS. Conversely, the slow-release rate from PC could be due to the additional physical crosslinking between the zwitterionic phosphorylcholine moieties, which further restrain the release of OVA from the hydrogel networks. PCDX and CMDX experienced much slower degradation rate than DX and DEDX. DX, and DEDX were completely degraded and released all protein cargoes by day 7. Meanwhile, CMDX and PCDX achieved 65% and 48% cumulative release of OVA. When both hydrogel MPs were treated with 0.1% V/V dextranase for 20 minutes at 37° C. to completely digest the MPs, almost all OVA can be recovered. Indeed, it has been reported that the anionic carboxylate moieties in CMDX could reduce the hydrolytic rate of the polymer chains [38]. For PCDX hydrogel, the zwitterionic groups could further influence the local gel environment, resulting in lower degradation rate of the polymer chains than CMDX.

MPs demonstrated good cell compatibility with >75% viability in PI stain on JAWSII culture (FIG. 3C). The released OVA from MPs was internalized by cells and the antigen presentations on MHC molecules were examined with fluorescent antibodies staining and flow cytometry at 24-hour. In FIG. 3D, JAWSII was treated with the same amount of F-OVA in all groups; for instance the cells were treated with bolus OVA in TCP and LPS groups whereas OVA in CMDX, DEDX, DX and PCDX were encapsulated and released from MPs.

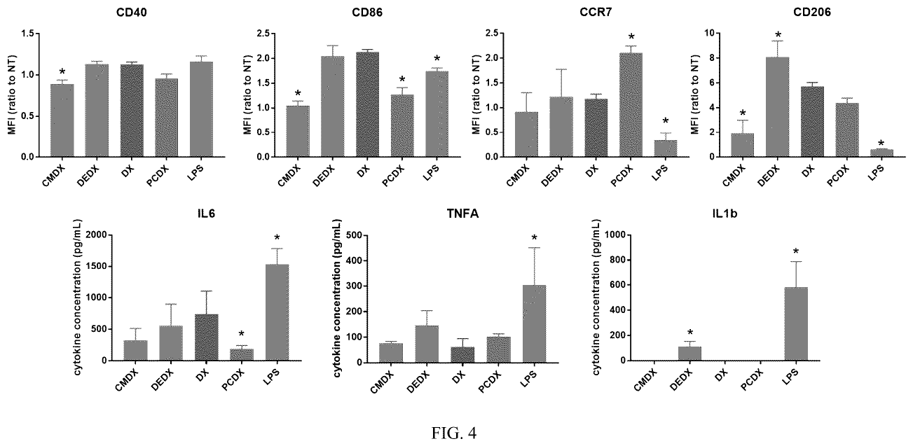

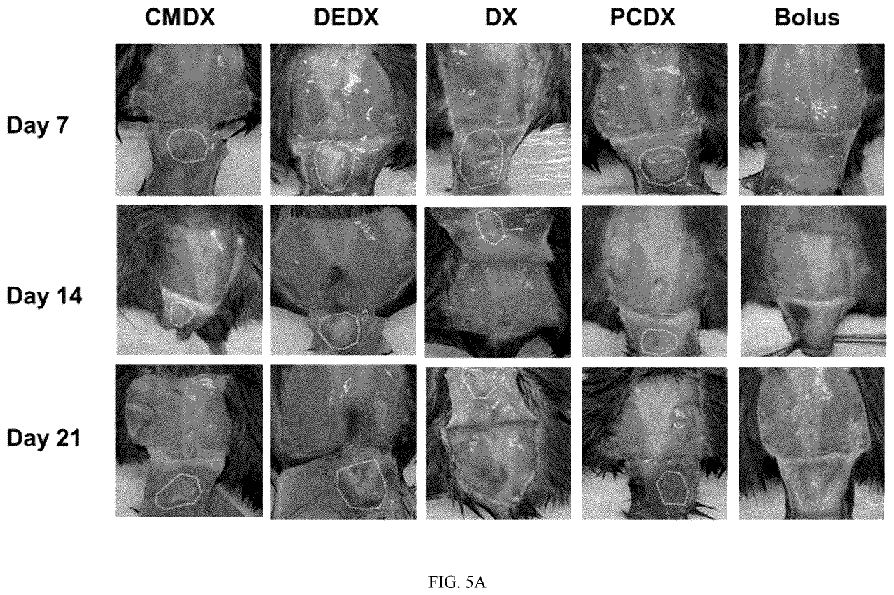

In this study, the F-OVA uptake trend was CMDX>DEDX>DX=PCDX, whereas the cumulative amount of F-OVA release from MPs in FIG. 3B was DEDX=DX>CMDX>PCDX by day 1. Despite of lower F-OVA release rate, CMDX showed higher F-OVA uptake than DEDX and DX could be due to the biomaterial properties in suppressing JAWSII maturation as shown in FIG. 4 in vitro phenotype profile. On the other hand, the antigen uptake levels for DEDX, DX and LPS+bolus OVA groups were significantly suppressed despite of higher antigen exposure on day 1. Such observation was consistent to previously reported results, of which JAWSII cultured on DX hydrogel surface had lower soluble antigen uptake than cells on hydrogels made of anionic hyaluronic acid or cationic glycol chitosan. Moreover, the pro-inflammatory environment in DX, DEDX and LPS could also promote DC maturation and reduction in antigen uptake capacity. Without being bound to any theory, the effect of biomaterials on cellular response could be due to the competitive engagement of the mannose receptors, i.e., CD206, by the interconnected DX polymer networks and led to lower antigen internalization [39-40]. However, such trend was reversed when DX backbones were modified with anionic carboxyl groups in CM and zwitterionic phosphorylcholine moieties in PC.

In terms of antigen presentation on MHC molecules in FIGS. 3E-3F, we observed higher MHC-I in PCDX and MHC-II in DEDX than other hydrogel groups. PCDX showed weaker induction in RAW264.7 in terms of lower cell adhesion tendency, activation markers (CCR7, CD86 and CD206) and IL-6, TNF-a levels in vitro culture, as shown in FIG. 2. Conversely on JAWSII effect, PCDX could engage JAWSII adhesion and immunogenic maturation but with lower levels of CD86 surface marker, and cytokines IL-6, TNF-α, and IL-1β in FIG. 4. Nonetheless, in FIG. 3F, JAWSII with PCDX exhibited significantly higher MHC-I SIINFEKL levels than other hydrogel groups and was comparable to cells cultured on TCP exposed to bolus OVA.

This could be due to the timing of DC maturation and antigen cross-presentation. It was reviewed that the cross-presentation increases in semi-mature DC and decreases in fully maturation state [41]. In this study, we observed that cells with PCDX and CMDX showed significantly higher antigen presentation on MHC-I than other hydrogel groups. PCDX demonstrated comparable MHC-I level as cells on TCP. Without being bound to any theory, we speculated that both PCDX and CMDX could induce higher MHC-I due to suppression of JAWSII maturation by the hydrogel carriers. Similarly, DEDX, DX and LPS which strongly induced DC maturation, resulted in lower MHC-I antigen cross-presentation.

Furthermore, antigen cross-presentation involves early endosomes in recycling antigen-MHC-I complex back to cell surface, whereas lysosomal/late endosomal products are often subjected to MHC-II pathways [42]. In this study, we investigated the intracellular trafficking of antigens with LysoTracker™ Red DND-99 which stains intracellular acidic compartments, such as late endosomes and lysosomes. Our results in FIG. 3A shows that CMDX and PCDX engaged significantly less lysosomal activity than DEDX and DX, implicating higher likelihood of MHC-I recycling in early endosomes and antigen cross-presentation. Learning from the immune activation mechanisms of cationic polysaccharides and delivery particles coated with cationic entities, we infer that the cationic moieties on DEDX could promote TLR4 engagement as LPS, leading to similar immunogenic activation profile [43-45].

### Example 4—MPs Effects on Cell Immune Phenotypes In Vitro

We investigated the effect of MPs in stimulating DC maturation profile in vitro. In terms of pro-inflammatory markers, CD40 and CD86 are co-stimulatory molecules presented on mature immunogenic DCs to positively induce T-cell differentiations. IL-6, TNF-α and IL-1β are pro-inflammatory cytokines that signify the immunogenic maturation of DCs. As shown in FIG. 4, we observed PCDX and CMDX suppressed the pro-inflammatory maturation of JAWSII with lower expression of CD86 and CD40. Additionally, the pro-inflammatory cytokines, TNF-α, IL-1β and IL-6 levels were also lower for both hydrogel MPs.

Besides the co-stimulatory markers, we also studied the expression levels of CCR7, representing the DC homing to lymph nodes in achieving successful induction of adaptive immune responses. We observed than PCDX could significantly induced highest expression level of CCR7 amongst MPs, whereas the modification of DX with anionic and cationic groups caused no significant change on JAWSII (FIG. 4). Conversely, LPS significantly suppressed CCR7 expression, which is consistent to previously reported results [13].

### Example 5-Zwitterionic MPs Suppressed FBR, while Engaging CD11c⁺ Cells In Vivo

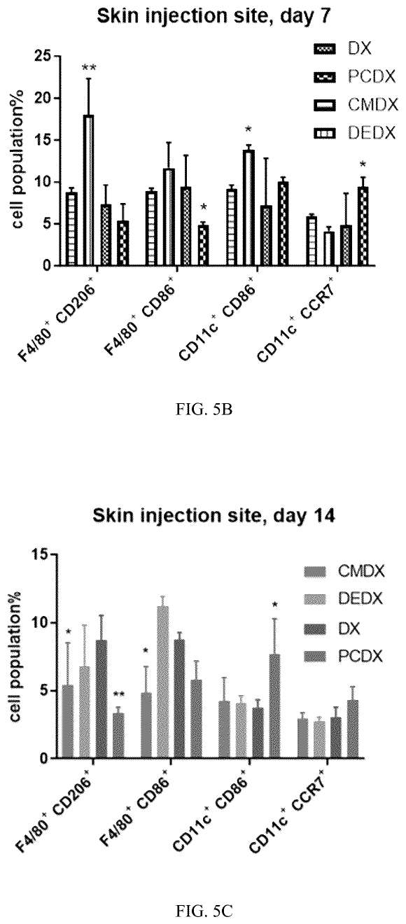

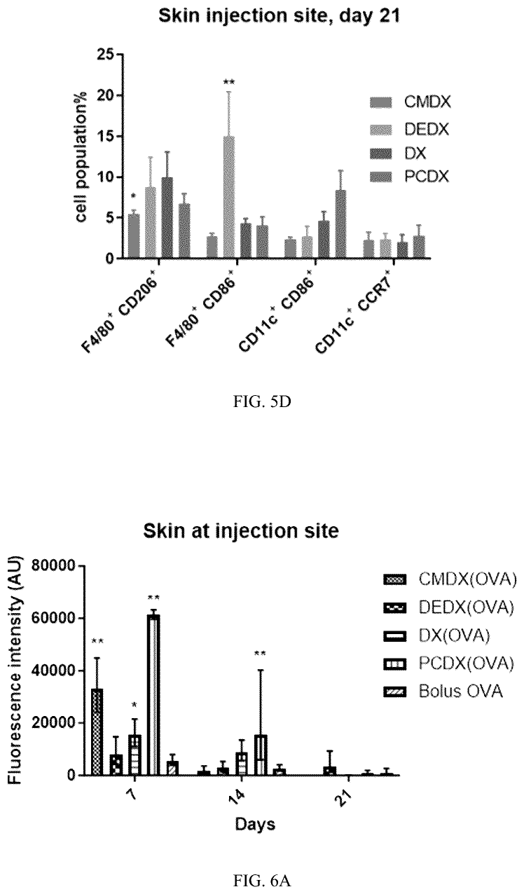

Next, we further examined the tissue environment at the MPs injection sites in immunocompetent C57BL\6 mice. We monitored the development of fibrotic tissues at the injection sites with time. We observed there is severe development of FBR, signified by fibrosis and angiogenesis at the injection site in DEDX and DX from day 7 to 21, as shown in FIG. 5A. Conversely, there was no FBR in PCDX and CMDX MPs, due to the anti-fouling properties of biomaterials as also observed in animals treated with bulk hydrogels and in vitro cell results [46].

In FIGS. 5B-D, we observed dynamic population change surrounding the injected hydrogel MPs with time. It was found that the cell populations surrounding DEDX MPs were majorly F4/80⁺ macrophages at CD206⁺ (M2 state) in the initial timepoint. F4/80⁺ CD86⁺ became dominant on day 14 and 21. Such population change could be due to the release of soluble cationic DEDX degradation products as the MPs degrade with time, resulting in pro-inflammatory local microenvironment and promoting M1 macrophage activation. On the other hand, the anti-fouling properties of PCDX showed significant suppression of macrophage population activation and enhanced CD11c⁺ with enhanced CCR7 expression at the initial timepoint, and persistently higher level of CD86 until day 21. For the case of CMDX, despite of its low fouling properties as examined in vitro and in vivo, the tissue environment surrounding the MP injection site revealed lower CD11c⁺ and F4/80⁺ cells engagement as compared to other hydrogel groups.

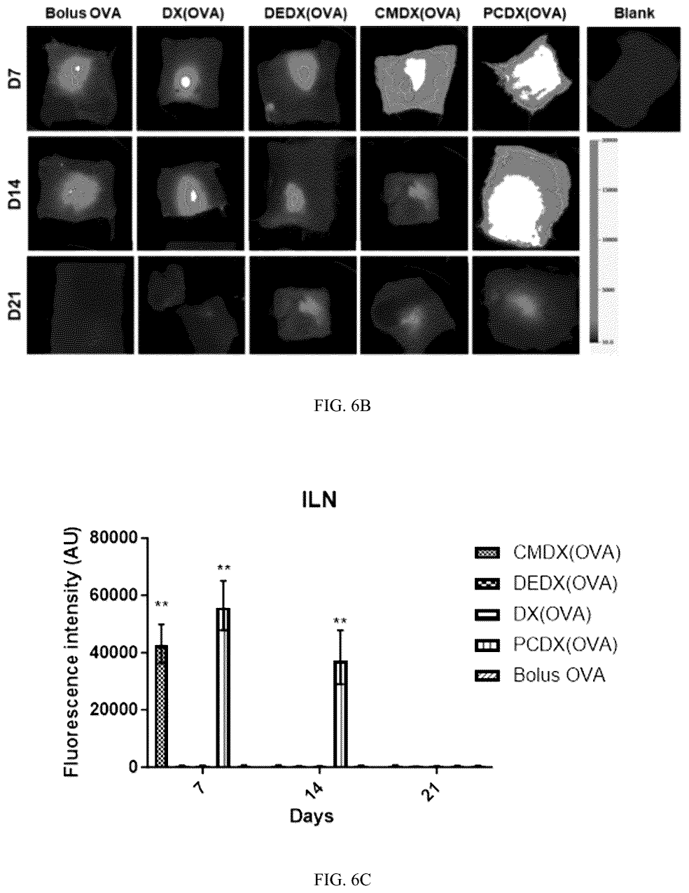

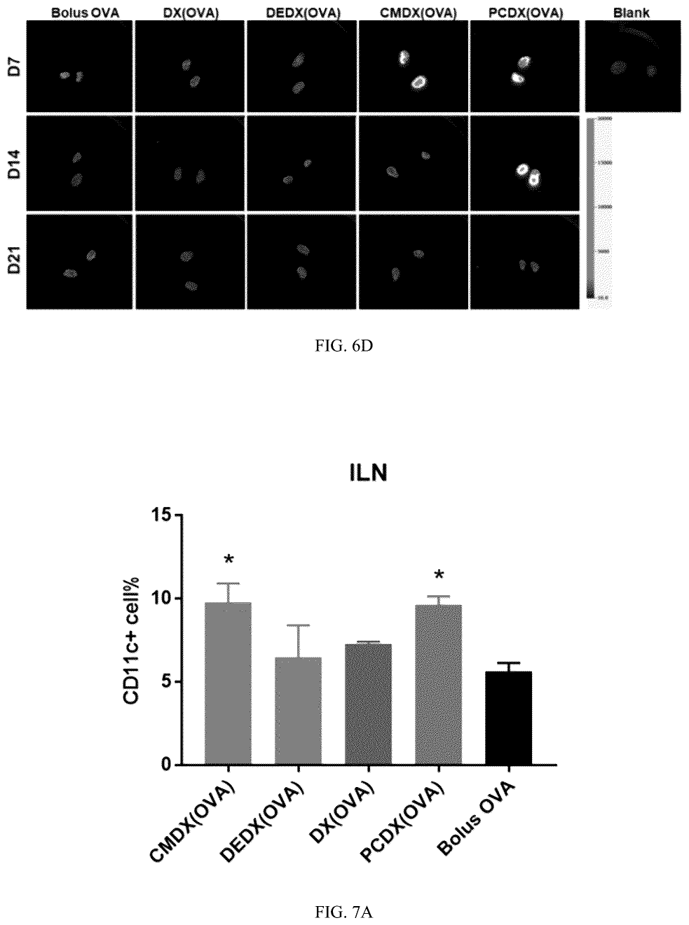

We also examined the effect of modification on the biodistribution of infrared-probed OVA antigen delivered from the MPs depot in vivo, particularly at the subcutaneous injection site and inguinal lymph nodes. The results in FIGS. 6A-6B revealed that the antigen signals in skin remained significantly higher on day 7 and 14 for PCDX. On the other hand, DEDX and DX demonstrated similar trend as animals treated with bolus OVA, with antigen signals beginning to diminish on day 7 and 14. Without being bound to any theory, the sustained release of antigen from PCDX could be due to the slow-release rate and degradation rate of MPs as well as low FBRs, resulting in slower release of antigens to the target immune cells at the injection sites, e.g., dendritic cells on day 14. Indeed, we observed higher antigen signals mobilized to ILN of PCDX groups on day 7 and 14 than other MPs groups (FIG. 6C-6D). Conversely, the significant reduction of antigen signals in DX and DEDX from day 7 could be due to the rapid antigen clearance from recruited macrophages and other phagocytic cells in FBR cascades, as observed in FIG. 5B. Nonetheless in contrast to PCDX, the antigen signal in skin of CMDX experienced significant drop by day 14. Such drastic signal reduction could be due to the rapid release and clearance of antigen signals to the systemic system after day 7 when the MPs were completely degraded and released the payloads. Moreover, the antigen signals in ILN in CMDX also significantly decreased on day 14. By day 21, all groups demonstrated similarly low level of antigen signals at the injection site.

### Example 6—Zwitterionic MPs Promoted Antigen Specific Cellular and Humoral Responses In Vivo

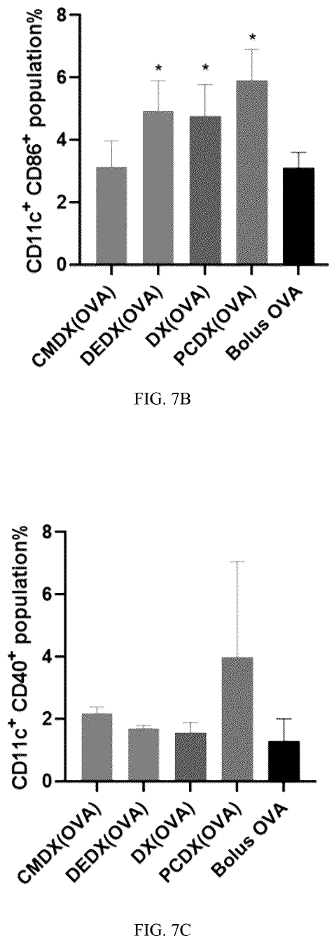

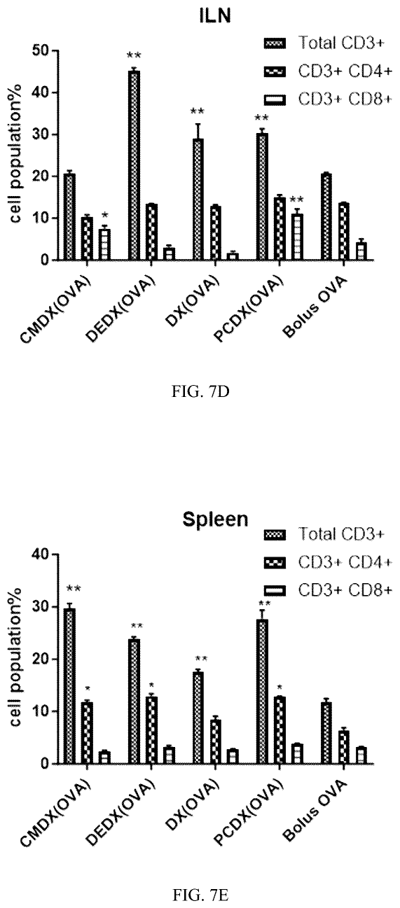

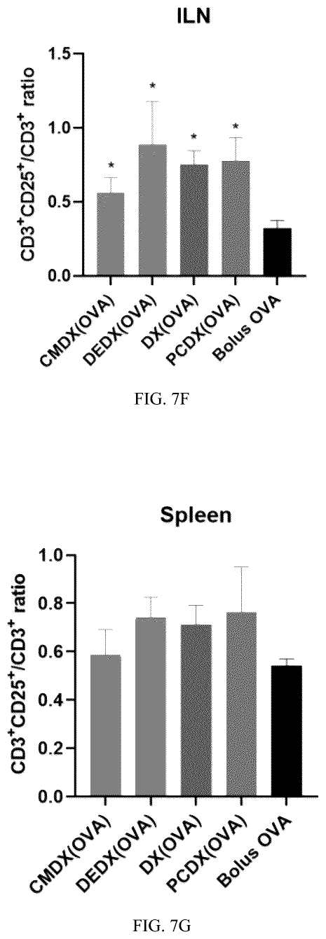

Following the biodistribution of antigens delivered with MPs, we examined the population distribution of migratory dendritic cells and lymphocytes in secondary lymphoid organs, such as ILN and spleen of C57BL\6 mice. Consistent to the antigen signals mobilization to lymph nodes in FIGS. 6C-6D on day 7, we observed significantly higher percentage of CD11c⁺ cells in ILN of CMDX and PCDX treated animals than bolus OVA treatment (FIG. 7A).

Furthermore, we investigated the activation profile of DC in ILN, namely CD86 and CD40, both of which are positive co-stimulatory molecules on antigen presenting cells in stimulating T-cells activation and differentiation. Interestingly, despite of the lower immunogenic profile of PCDX in the in vitro JAWSII study in FIG. 4, the in vivo study results revealed that PCDX displayed similar CD86+ population frequency of 61.4% in CD11c⁺ cells as compared DX with 65.6% of CD86+ cells in CD11c⁺ in ILN in FIG. 7B. This suggests the potential adjuvanting ability of PCDX backbone as the hydrogel degrades and triggers stronger immune responses in vivo. The CD86+ frequency of CD11c⁺ parent was 32.1% and 96.4% for CMDX and DEDX, respectively. On the other hand, the level of CD11c⁺, CD40+ cells were also greater in PCDX, but the difference was insignificant as compared to bolus and other hydrogel treatments. Such results indicate that by circumventing fibrotic events with PCDX at the hydrogel injection sites, the resident CD11c⁺ DC could be engaged by the local extended release of antigens. As a result, more activated resident DC carrying the antigen and presenting co-stimulatory molecules could be mobilized to nearby ILN to trigger naïve lymphocytes differentiation.

In terms of lymphocytes profile, PCDX induced significantly higher level of CD3+ cells than bolus and CMDX, whereby the level of CD3+CD8+ cells in ILN were significantly higher in PCDX at an average of 11.07% than other MP groups. Conversely, the level of CD3+ CD4+ cells of PCDX were similar to MPs and bolus (FIG. 7E). Notably, DEDX could induce the highest total percentage of CD3+ cells amongst all treatment groups, whereas CMDX lymphocytes profile in ILN were similar to that of bolus group.

For spleen lymphocytes, the average level of CD3+ cells for PCDX, CMDX, and DEDX were 29.7%, 23.83%, and 27.67% respectively. They were all significantly higher than bolus with an average value of 11.7%. Furthermore, PCDX, CMDX, and DEDX were also able to induce on average two times higher CD3+ CD4+ cells of 11.73%, 12.9%, and 12.7% as compared to bolus with 6.4% CD4+ cells. Unlike ILN lymphocytes profile, spleen exhibited similar induction of CD3+ CD8+ cells for all treatment groups. They were 2.4%, 3.2%, 2.8%, and 3.8% for CMDX, DEDX, DX, and PCDX respectively, in comparison to bolus that was averagely 2.9%. Notably, the soluble zwitterionic polysaccharides were known to induce MHC-II responses and CD4+ T-cell responses [47]. Here we demonstrated that the zwitterionic PCDX MPs particularly were able to induce both MHC-I and II responses, resulting in the activation of both CD4+ and CD8+ arms of T-cells in ILN and spleen.

In terms of T-cell activation level in vivo, we examined the population frequency of CD3+ CD25+ in ILN and spleen in FIGS. 7F-7G. The results revealed that T-cell activation by all hydrogel groups were significantly higher than bolus OVA treatment, suggesting the advantage of extended antigen release in triggering stronger T-cell response. As expected from the DC (CD86 and CD40) profile in the ILN on day 7, the level of ILN T-cell activation was the highest in DEDX, followed by DX and PCDX, then CMDX, and bolus OVA. Similar trend of T-cell activation was seen in spleen, but the difference was not significant.

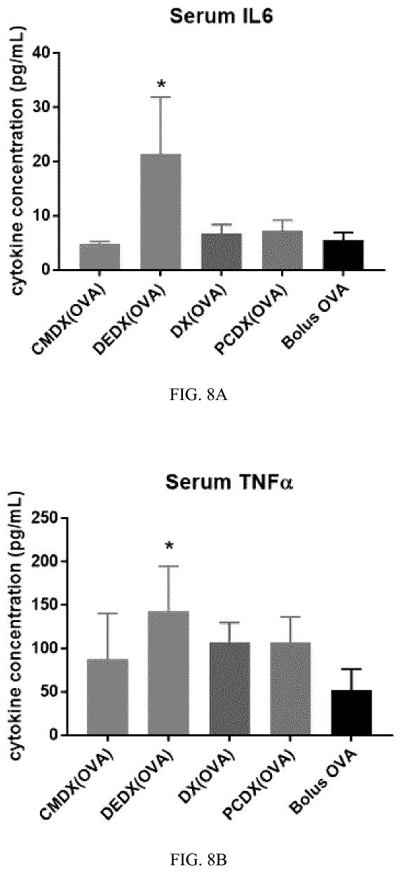

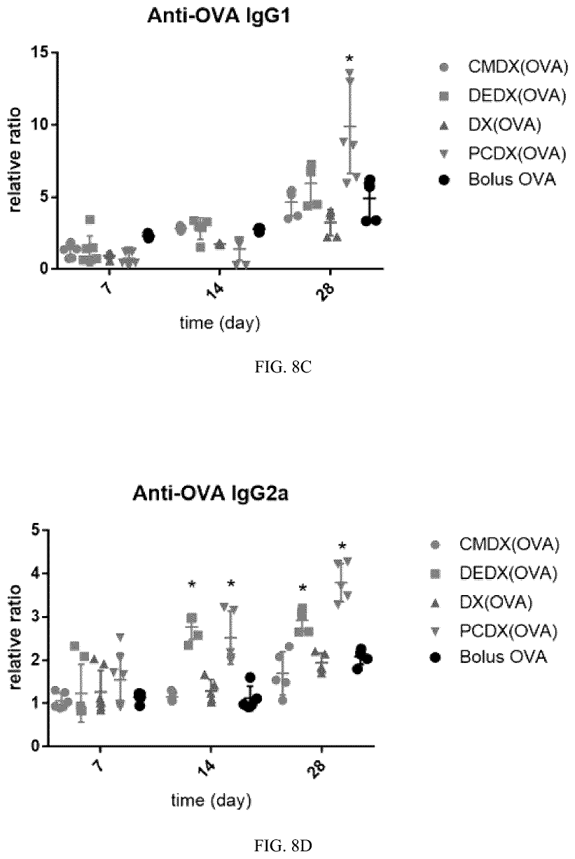

In terms of serum cytokine profile in FIGS. 8A-8B, we identified that PCDX induce similar level of IL-6 and TNF-α as bolus, DX and CMDX on day 7 post-injection. DEDX conversely was able to induce the most prominent systemic pro-inflammatory response with significantly elevated level of serum IL-6 and TNF-α, at 20 μg/mL and 148 μg/mL higher than other treatment groups, respectively. In addition, we also monitored the humoral response of animals treated with MPs. We observed PCDX MPs were able to induce greater humoral response rate than bolus and DX in terms of higher amount of IgG1 and IgG2a levels in serum at day 28 (FIGS. 8C-8D), implicating greater extent of Th2 and Th1 responses compared to other treatment groups.

Here we demonstrated polysaccharide hydrogels could serve dual roles: (1) antigen depot for sustained release of antigens, and (2) self-adjuvanticity to interact and activate local APCs. Particularly, the zwitterionic PCDX hydrogel microparticles poses as effective vaccine delivery system in providing therapeutic cancer vaccination and immunity against intracellular infections. In comparison to the conventional bolus administration of antigens, the hydrogel carrier could encapsulate antigens and protect them from degradation. The controlled release properties of hydrogel also enabled extended and spatial release of antigen loads at the subcutaneous injection sites to modulate resident innate immune cells, such as DC and macrophages in situ. In comparison to other hydrogel carrier, the incorporation of zwitterionic tandem groups on the polysaccharide backbone could help to suppress tissue fibrosis around the device. In this study, the in vitro and in vivo foreign body assays revealed that PCDX microparticle system could more effectively inhibit macrophage and fibrotic cells adhesion, suppress macrophage-mediated clearance, and circumvent the formation of fibrotic capsules surrounding the hydrogel device post-subcutaneous injection. As a result, PCDX could help extending the bioavailability of antigens for adaptive immunity induction. Furthermore, the DX-based carriers could selectively engage the immunogenic maturation of resident DCs to further enhance the immune response of vaccines. The in vivo evaluation revealed that such hydrogel delivery system induced adaptive cellular and humoral responses that are favorable for anti-cancer immunity. In contrast to other hydrogel systems, PCDX induced significantly higher CD8 T-cell populations in ILN and spleen, as well as higher IgG2a titer.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLARY EMBODIMENTS

Embodiment 1. A zwitterionic compound of formula (I), Formula (I)

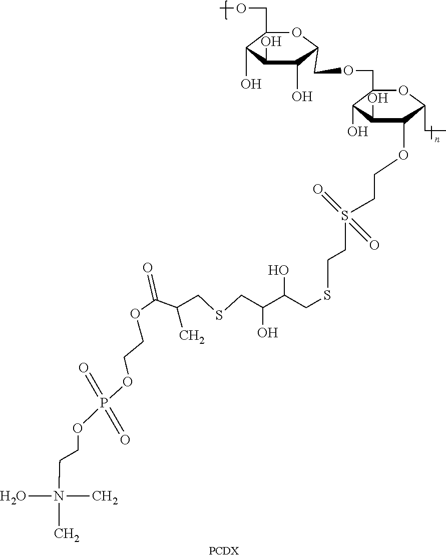

wherein the zwitterionic compound is PCDX.

Embodiment 2. The zwitterionic compound of embodiment 1, wherein the compound is synthesized from the conjugation of phosphorylcholine moieties with chemically modified DX, wherein the chemically modified DX comprises the forms of aldehyde, vinyl sulfone, acrylate, methacrylate, at various degree of modifications (DM) ranging from about 1 to about 20%.

Embodiment 3. The zwitterionic compound of embodiment 1, wherein the zwitterionic compound PCDX is functionalized with pendant crosslinking moieties selected from the group consisting of acrylate, methacrylate, vinyl sulfone, aldehyde, amine and thiol groups at various degree of modifications ranging from about 1 to about 20% to obtain an activated hydrogel precursor of PCDX, wherein the PCDX precursor is crosslinked to form an injectable PCDX hydrogel.

Embodiment 4. A hydrogel depot, comprising the injectable PCDX hydrogel of embodiment 3, wherein the hydrogel depot is selected from the group consisting of bulk hydrogel and hydrogel microparticles (MPs).

Embodiment 5. An immunomodulating polysaccharide-based depot system, comprising the hydrogel depot of embodiment 4, wherein the hydrogel depot is used as a carrier comprising vaccine components, wherein the vaccine components comprise one or more antigens, and wherein the hydrogel is injected under the skin of a subject for the sustained release of the one or more antigens to induce effective immune responses in the subject.

Embodiment 6. The immunomodulating polysaccharide-based depot system of embodiment 5, wherein the subject is a mammal, and the mammal is a human.

Embodiment 7. The immunomodulating polysaccharide-based depot system of any of the preceding embodiments, wherein the one or more antigens comprise a protein, a nucleic acid, a sugar, a lipid, or a glycoprotein, or any combinations thereof.

Embodiment 8. The immunomodulating polysaccharide-based depot system of any of the preceding embodiments, wherein the zwitterionic moieties of PCDX reduce rapid hydrogel swelling and provide slow antigen release.

Embodiment 9. The immunomodulating polysaccharide-based depot system of any of the preceding embodiments, wherein a MP has a diameter that ranges from about 2 to about 500 μm.

Embodiment 10. The immunomodulating polysaccharide-based depot system of any of the preceding embodiments, wherein a MP has a diameter that ranges from about 2 to about 500 μm.

Embodiment 11. The immunomodulating polysaccharide-based depot system embodiment 10, wherein the MP has a diameter of about 72.4±3.1 μm.

Embodiment 12. The immunomodulating polysaccharide-based depot system of any of the preceding embodiments, wherein the hydrogel depot is used as an antigen depot and as a self adjuvant.

Embodiment 13. The immunomodulating polysaccharide-based depot system of any of the preceding embodiments, wherein both the injectable scaffold and microparticles (MPs) forms of the hydrogel depot effectively evade FBR when injected under the skin of a subject.

Embodiment 14. The immunomodulating polysaccharide-based depot system of any of the preceding embodiments, wherein the PCDX hydrogel significantly induces the expression of markers selected from the group consisting of DCs markers CD86 and CD40, lymph node marker CCR7, mannose receptor CD206, pro-inflammatory markers CD80/CD83, MHC-II, MHC-I, and secreted cytokines IFN-g, TNF-a, IL-6, and IL-1b.

Embodiment 15. A method for extended vaccine delivery, comprising injecting subcutaneously in a subject an effective amount of a MP comprising a PCDX hydrogel, one or more antigens, and optionally an excipient, wherein the MP provides the sustained release of the one or more antigens to induce effective immune responses in the subject.

Embodiment 16. The method for extended vaccine delivery of embodiment 15, wherein the subject is a mammal, and the mammal is a human.

Embodiment 17. The method for extended vaccine delivery of any of the preceding embodiments, wherein the one or more antigens comprise a protein, a nucleic acid, a sugar, a lipid, or a glycoprotein, or any combinations thereof.

Embodiment 18. The method for extended vaccine delivery of any of the preceding embodiments, wherein the MP has a diameter that ranges from about 2 to about 500 μm.

Embodiment 19. The method for extended vaccine delivery of any of the preceding embodiments, wherein the MP has a diameter that ranges from about 68 to about 80 μm.

Embodiment 20. The method for extended vaccine delivery of embodiment 19, wherein the MP has a diameter of about 72.4±3.1 μm.

Embodiment 21. The method for extended vaccine delivery of any preceding of the embodiments, wherein the MP is used as an antigen depot and as a self adjuvant.

Embodiment 22. The method for extended vaccine delivery of any of the preceding embodiments, wherein the MP effectively evade FBR when injected under the skin of a subject.

Embodiment 23. The method for extended vaccine delivery of any of the preceding embodiments, wherein the MP significantly induces the expression of markers selected from the group consisting of DCs markers CD86 and CD40, lymph node marker CCR7, and mannose receptor CD206.

### REFERENCES

1. Sabado R L, Balan S, Bhardwaj N. Dendritic cell-based immunotherapy. Cell Res. 2016; 27 (1): 74-95.
2. Fucikova J, Palova-Jelinkova L, Bartunkova J, Spisek R. Induction of tolerance and immunity by dendritic cells: Mechanisms and clinical applications. Front Immunol. 2019; 10 (2393): 1-17.
3. Cabeza-Cabrerizo M, Cardoso A, Minutti C M, Pereira Da Costa M, Reis E Sousa C. Dendritic Cells Revisited. Annu Rev Immunol. 2021; 39:131-66.
4. Segura E. Review of mouse and human dendritic cell subsets. Methods in Molecular Biology. 2016; 1423:3-15.
5. Macri C, Dumont C, Johnston A P R, Mintern J D. Targeting dendritic cells: a promising strategy to improve vaccine effectiveness. Clin Transl Immunology. 2016; 5 (3):e66-8.
6. Lehmann C H K, Heger L, Heidkamp G F, Baranska A, Lühr J J, Hoffmann A, et al. Direct Delivery of Antigens to Dendritic Cells via Antibodies Specific for Endocytic Receptors as a Promising Strategy for Future Therapies. Vaccines. 2016; 4 (2): 8
7. De Gregorio E, Caproni E, Ulmer J B. Vaccine adjuvants: Mode of action. Front Immunol. 2013; 4 (July): 214.
8. Awate S, Babiuk L A, Mutwiri G. Mechanisms of action of adjuvants. Front Immunol. 2013; 4 (MAY): 114.
9. Moyer T J, Zmolek A C, Irvine D J. Beyond antigens and adjuvants: formulating future vaccines. J Clin Invest. 2016; 126 (3): 799.
10. Asai D, Fukuda T, Morokuma K, Funamoto D, Yamaguchi Y, Mori T, et al. Injectable Polypeptide Hydrogel Depot System for Assessment of the Immune Response-Inducing Efficacy of Sustained Antigen Release Alone. Macromol Biosci. 2019; 19 (10): 1900167.
11. Ali O A, Huebsch N, Cao L, Dranoff G, Mooney D J. Infection-mimicking materials to program dendritic cells in situ. Nat Mater. 2009; 8 (2): 151-8.
12. Park J, Babensee J E. Differential functional effects of biomaterials on dendritic cell maturation. Acta Biomater. 2012; 8 (10): 3606-17.
13. Chung J T, Lau CML, Chau Y. The effect of polysaccharide-based hydrogels on the response of antigen-presenting cell lines to immunomodulators. Biomater Sci. 2021; 9 (19): 6542-54.
14. He T, Liang X, Li L, Gong S, Li X, Zhang M, et al. A spontaneously formed and self-adjuvanted hydrogel vaccine triggers strong immune responses. Mater Des. 2021; 197:109232.
15. Sheikh Z, Brooks P J, Barzilay O, Fine N, Glogauer M. Macrophages, foreign body giant cells and their response to implantable biomaterials. Materials. 2015; 8 (9): 5671-701.
16. Fayzullin A, Bakulina A, Mikaelyan K, Shekhter A, Guller A. Implantable drug delivery systems and foreign body reaction: Traversing the current clinical landscape. Bioengineering. 2021; 8 (12): 205.
17. Mariani E, Lisignoli G, Borzì R M, Pulsatelli L. Biomaterials: Foreign bodies or tuners for the immune response? Int J Mol Sci. 2019; 20 (3): 636.
18. Yu Y, Chau Y. One-step “click” method for generating vinyl sulfone groups on hydroxyl-containing water-soluble polymers. Biomacromolecules. 2012; 13 (3): 937-42.
19. Lau CML, Jahanmir G, Yu Y, Chau Y. Controllable multi-phase protein release from in-situ hydrolyzable hydrogel. Journal of Controlled Release. 2021; 335:75-85.
20. H. Y. Chung C, Ming Laurence Lau C, T. Sin D, Teng Chung J, Zhang Y, Chau Y, et al. Droplet-Based Microfluidic Synthesis of Hydrogel Microparticles via Click Chemistry-Based Cross-Linking for the Controlled Release of Proteins. ACS Appl Bio Mater. 2021; 4 (8): 6186-94.
21. Pustylnikov S, Sagar D, Jain P, Khan Z K. Targeting the C-type lectins-mediated host-pathogen interactions with dextran. J Pharm Pharm Sci. 2014; 17 (3): 371-92.
22. Shen L, Higuchi T, Tubbe I, Voltz N, Krummen M, Pektor S, et al. A Trifunctional Dextran-Based Nanovaccine Targets and Activates Murine Dendritic Cells, and Induces Potent Cellular and Humoral Immune Responses In Vivo. PLOS One. 2013; 8 (12):e80904.
23. Goreish H H, Lewis A L, Rose S, Lloyd A W. The effect of phosphorylcholine-coated materials on the inflammatory response and fibrous capsule formation: In vitro and in vivo observations. J Biomed Mater Res A. 2004; 68A (1): 1-9.
24. Iwasaki Y, Ishihara K. Phosphorylcholine-containing polymers for biomedical applications. Anal Bioanal Chem. 2005; 381 (3): 534-46.
25. Demento S L, Siefert A L, Bandyopadhyay A, Sharp F A, Fahmy T M. Pathogen-associated molecular patterns on biomaterials: a paradigm for engineering new vaccines. Trends Biotechnol. 2011; 29 (6): 294-306.
26. Oakes R S, Froimchuk E, Jewell C M. Engineering Biomaterials to Direct Innate Immunity. Adv Ther (Weinh). 2019; 2 (6): 1800157.
27. Baranov M V., Kumar M, Sacanna S, Thutupalli S, van den Bogaart G. Modulation of Immune Responses by Particle Size and Shape. Front Immunol. 2021; 11:3854.
28. Singh A, Peppas N A. Hydrogels and scaffolds for immunomodulation. Adv Mater. 2014; 26 (38): 6530.
29. Joyce J C, Sella H E, Jost H, Mistilis M J, Esser E S, Pradhan P, et al. Extended delivery of vaccines to the skin improves immune responses. J Control Release. 2019; 304:135.
30. Saxena R K, Vallyathan V, Lewis D M. Evidence for lipopolysaccharide-induced differentiation of RAW264.7 murine macrophage cell line into dendritic like cells. J Biosci. 2003; 28 (1): 129-234.
31. Jansen L E, Amer L D, Chen EYT, Nguyen T V., Saleh L S, Emrick T, et al. Zwitterionic PEG-PC hydrogels modulate the foreign body response in a modulus-dependent manner. Biomacromolecules. 2018; 19 (7): 2880.
32. Yu T, Wang W, Nassiri S, Kwan T, Dang C, Liu W, et al. Temporal and spatial distribution of macrophage phenotype markers in the foreign body response to glutaraldehyde-crosslinked gelatin hydrogels. J Biomater Sci Polym Ed. 2016; 27 (8): 721.
33. Kou P M, Babensee J E. Macrophage and dendritic cell phenotypic diversity in the context of biomaterials. J Biomed Mater Res A. 2011; 96 A (1): 239-60.
34. Anderson J M, Rodriguez A, Chang D T. Foreign body reaction to biomaterials. Semin Immunol. 2008; 20 (2): 86-100.
35. Yu Y, Lau L C M, Lo A C yin, Chau Y. Injectable Chemically Crosslinked Hydrogel for the Controlled Release of Bevacizumab in Vitreous: A 6-Month In Vivo Study. Transl Vis Sci Technol. 2015; 4 (2): 5.
36. Chung C H Y, Lau C M L, Sin D T, Chung J T, Zhang Y, Chau Y, et al. Droplet-Based Microfluidic Synthesis of Hydrogel Microparticles via Click Chemistry-Based Cross-Linking for the Controlled Release of Proteins. ACS Appl Bio Mater. 2021; 4 (8): 6186-94.
37. Johnston S A, May R C. Cryptococcus interactions with macrophages: evasion and manipulation of the phagosome by a fungal pathogen. Cell Microbiol. 2013; 15 (3): 403-11.
38. Lau C M L, Jahanmir G, Chau Y. Local environment-dependent kinetics of ester hydrolysis revealed by direct 1H NMR measurement of degrading hydrogels. Acta Biomater. 2020; 101:219-26.
39. Burgdorf S, Lukacs-Kornek V, Kurts C. The Mannose Receptor Mediates Uptake of Soluble but Not of Cell-Associated Antigen for Cross-Presentation. The Journal of Immunology. 2006; 176 (11): 6770-6.
40. Pustylnikov S, Sagar D, Jain P, Khan Z K. Targeting the C-type lectins-mediated host-pathogen interactions with dextran. Journal of Pharmacy and Pharmaceutical Sciences. 2014; 17 (3): 371-92.
41. Alloatti A, Kotsias F, Magalhaes J G, Amigorena S. Dendritic cell maturation and cross-presentation: timing matters! Immunol Rev. 2016; 272 (1): 97-108.
42. Embgenbroich M, Burgdorf S. Current Concepts of Antigen Cross-Presentation. Front Immunol. 2018; 9:1643.
43. Zhang P, Liu W, Peng Y, Han B, Yang Y. Toll like receptor 4 (TLR4) mediates the stimulating activities of chitosan oligosaccharide on macrophages. Int Immunopharmacol. 2014; 23 (1): 254-61.
44. Mulens-Arias V, Rojas J, Pérez-Yagüe S. Polyethylenimine-coated SPIONs trigger macrophage activation through TLR-4 signaling and ROS production and modulate podosome dynamics. Biomaterials. 2015; 52:494-506.
45. Zapala L, Drela N, Bil J, Nowis D, Basak G W, Lasek W. Optimization of activation requirements of immature mouse dendritic JAWSII cells for in vivo application. Oncol Rep. 2011; 25 (3): 831-40.
46. Chung, J. T., Lau, C. M. L., Chung, C. H., Rafiei, M., Yao, S., & Chau, Y. (2023). Vaccine delivery by zwitterionic polysaccharide-based hydrogel microparticles showing enhanced immunogenicity and suppressed foreign body responses. Biomaterials Science, 11 (14), 4827-4844.
47. Kalka-Moll W M, Tzianabos A O, Bryant P W, Niemeyer M, Ploegh H L, Kasper D L. Zwitterionic polysaccharides stimulate T cells by MHC class II-dependent interactions. J Immunol. 2002; 169 (11): 6149-53.

We claim:

1. A zwitterionic compound of formula I, Formula (I) 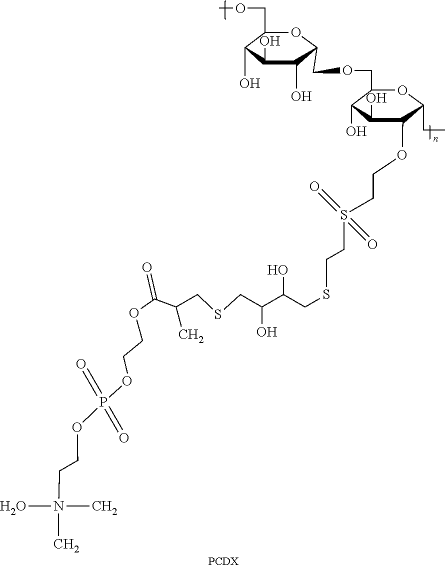
wherein the zwitterionic compound is PCDX.

2. The zwitterionic compound of claim 1, wherein the compound is synthesized from the conjugation of phosphorylcholine moieties with chemically modified DX, wherein the chemically modified DX comprises the forms of aldehyde, vinyl sulfone, acrylate, methacrylate, at various degree of modifications (DM) ranging from about 1 to about 20%.

3. The zwitterionic compound of claim 1, wherein the zwitterionic compound PCDX is functionalized with pendant crosslinking moieties selected from the group consisting of acrylate, methacrylate, vinyl sulfone, aldehyde, amine, and thiol groups at various degree of modifications ranging from about 1 to about 20% to obtain an activated hydrogel precursor PCDX, wherein the PCDX precursor is crosslinked to form an injectable PCDX hydrogel.

4. A hydrogel depot, comprising the injectable PCDX hydrogel of claim 3, wherein the hydrogel depot is selected from the group consisting of bulk hydrogel and hydrogel microparticles (MPs).

5. An immunomodulating polysaccharide-based depot system, comprising the hydrogel depot of claim 4, wherein the hydrogel depot is used as a carrier comprising vaccine components, wherein the vaccine components comprise one or more antigens, and wherein the hydrogel is injected under the skin of a subject for the sustained release of the one or more antigens to induce effective immune responses in the subject.

6. The immunomodulating polysaccharide-based depot system of claim 5, wherein the subject is a mammal, and the mammal is a human.

7. The immunomodulating polysaccharide-based depot system of claim 6, wherein the one or more antigens comprise a protein, a nucleic acid, a sugar, a lipid, a glycoprotein, or any combination thereof.

8. The immunomodulating polysaccharide-based depot system of claim 6, wherein the zwitterionic moieties of PCDX reduce rapid hydrogel swelling and provide slow antigen release.

9. The immunomodulating polysaccharide-based depot system of claim 6, wherein each MP has a diameter that ranges from about 2 to about 500 μm.

10. The immunomodulating polysaccharide-based depot system of claim 6, wherein a MP has a diameter that ranges from about 68 to about 80 μm.

11. The immunomodulating polysaccharide-based depot system of claim 10, wherein the MP has a diameter of about 72.4±3.1 μm.

12. The immunomodulating polysaccharide-based depot system of claim 6, wherein the hydrogel depot is used as an antigen depot and as a self adjuvant.

13. The immunomodulating polysaccharide-based depot system of claim 6, wherein both the injectable scaffold and microparticles (MPs) forms of the hydrogel depot effectively evade foreign body responses (FBR) when injected under the skin of a subject.

14. The immunomodulating polysaccharide-based depot system of claim 6, wherein the PCDX hydrogel significantly induces the expression of markers selected from the group consisting of DCs markers CD86 and CD40, lymph node marker CCR7, and mannose receptor CD206.

15. A method for extended vaccine delivery, comprising injecting subcutaneously in a subject an effective amount of a MP comprising a PCDX hydrogel, one or more antigens, and optionally an excipient, wherein the MP provides the sustained release of the one or more antigens to induce effective immune responses in the subject.

16. The method for extended vaccine delivery of claim 15, wherein the subject is a mammal, and the mammal is a human.

17. The method for extended vaccine delivery of claim 15, wherein the one or more antigens comprise a protein, a nucleic acid, a sugar, a lipid, or glycoprotein, or any combination thereof.

18. The method for extended vaccine delivery of claim 15, wherein the MP has a diameter that ranges from about 2 to about 500 μm.

19. The method for extended vaccine delivery of claim 18, wherein the MP has a diameter that ranges from about 68 to about 80 μm.

20. The method for extended vaccine delivery of claim 19, wherein the MP has a diameter of about 72.4±3.1 μm.

21. The method for extended vaccine delivery of claim 15, wherein the MP is used as an antigen depot and as a self adjuvant.

22. The method for extended vaccine delivery of claim 15, wherein the MP effectively evade FBR when injected under the skin of a subject.

23. The method for extended vaccine delivery of claim 15, wherein the MP significantly induces the expression of markers selected from the group consisting of DCs markers CD86 and CD40, lymph node marker CCR7, and mannose receptor CD206.
